(19) United States
(12) Patent Application Publication
(10) Pub. No.: US 2017/0228496 A1
(43) Pub. Date: Aug. 10, 2017

(54) SYSTEM AND METHOD FOR PROCESS CONTROL OF GENE SEQUENCING

(71) Applicant: ONTARIO INSTITUTE FOR CANCER RESEARCH, Toronto (CA)

(72) Inventors: Paul C. Boutros, Toronto (CA); Lauren Chong, Woodbridge (CA); Christopher Lalansingh, Markham (CA); Marco Albuquerque, Leamington (CA)

(21) Appl. No.: 15/329,037

(22) PCT Filed: Jul. 27, 2015
(86) PCT No.: PCT/CA2015/050710

§ 371 Date: Jan. 25, 2017

Related U.S. Application Data
(60) Provisional application No. 62/029,112, filed on Jul. 25, 2014.

(51) Int. Cl. G06F 19/22; C12Q 1/68; G06F 19/24

(52) U.S. Cl. CPC G06F 19/22; G06F 19/24; C12Q 1/6869; C12Q 1/6874

(57) ABSTRACT

Systems, methods and computer-readable media are provided for determining the amount of sequencing required to achieve a target sequencing quality of a genetic sample to be sequenced. The method comprises receiving a genetic sample and sequencing a portion of the genetic sample. A sequencing quality metric belonging to a category of sequencing quality metrics is generated from the sequencing. The amount of sequencing of the genetic sample required to achieve the target sequencing quality is determined by inputting the sequencing quality metric into a trained model. A system is also disclosed for genetic sequencing. Corresponding methods and computer-readable media are also provided.

### FIELD

The embodiments described herein relate generally to DNA sequencing techniques, and more particularly to systems, methods and devices for next-generation sequencing.

INTRODUCTION

Advances in DNA sequencing (next-generation sequencing, NGS) have made it practical and affordable for both research and commercial applications. NGS has enabled projects as diverse as elucidating evolutionary relationships¹⁻⁴, improving agricultural practices⁵⁻⁸, making pre-natal diagnoses⁹, estimating protein structure¹⁰ and examining disease etiologies¹¹⁻¹⁴. Some of the first routine commercial applications of NGS are likely to be in personalized medicine, where genomic profiles may predict treatment response^{15,16}.

As NGS increases in popularity, automated pipelines for processing large numbers of samples will be vital, particularly for whole genome sequencing (WGS). Since NGS studies suffer from biases introduced during library preparation¹⁷ and sequencing¹⁸, a key part of increased automation is quality control to ensure outputs meet pre-specified constraints.

A new, improved solution is thus needed for quality control in NGS to ensure outputs meet pre-specified constraints.

### SUMMARY

The present disclosure relates to systems and methods for determining the amount of sequencing required to achieve a target sequencing quality of a genetic sample to be sequenced, as well as systems and methods for genome sequencing.

In an aspect, there is disclosed a method of determining the amount of sequencing required to achieve a target sequencing quality of a genetic sample to be sequenced, the method comprising: receiving the genetic sample; sequencing a portion of the genetic sample; generating from the sequencing a sequencing quality metric, said sequencing quality metric belonging to a category of sequencing quality metrics; determining the amount of sequencing of the genetic sample required to achieve the target sequencing quality by inputting the sequencing quality metric into a model trained with a plurality of reference sequencing quality metrics to predict sequencing quality.

In another aspect, there is disclosed a system for genetic sequencing, the system comprising: a device for receiving a genetic sample; a device for sequencing one or more portions of the genetic sample; a device for capturing sequencing data; at least one processor configured to: generate signals for commencing sequencing the one or more portions of the genetic sample; during or after sequencing of the one or more portions, receiving sequencing data for the one or more portions; generating from the sequencing data, at least one sequencing quality metric, said at least one sequencing quality metric belonging to at least one category of sequencing quality metrics; and generating signals for continuing or aborting the sequencing of the same or additional portions of the genetic sample based on a determination of the amount of sequencing of the genetic sample required to achieve a target sequencing quality using said at least one sequencing quality metric and a model trained with reference sequencing quality metrics to predict sequencing quality.

In another aspect, there is disclosed a system for genome sequencing, the system comprising: a device for receiving a genome sample; a device for sequencing one or more portions of the genome sample; a device for capturing sequencing data; and at least one processor configured to perform the methods described herein.

In various further aspects, the disclosure provides corresponding systems and devices, and logic structures such as machine-executable coded instruction sets for implementing such systems, devices, and methods.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

In the figures, embodiments are illustrated by way of example. It is to be expressly understood that the description and figures are only for the purpose of illustration and as an aid to understanding.

Embodiments will now be described, by way of example only, with reference to the attached figures, described below.

FIG. 1 is a graph demonstrating cellularity of prostate tumour samples. All 27 tumour samples had values greater than 0.6, with a mean cellularity of 0.832 across samples.

FIG. 2 shows a summary of the sample data utilized in the present disclosure. Data was obtained from the CPC-GENE project and consisted of matched tumour and normal DNA from twenty-six prostate cancer patients (see Table 1), plus an additional unmatched tumour sample (CPCG0001P, not pictured). A library was created for each sample. For all tumour libraries, six lanes of DNA were sequenced. For normal libraries, four lanes of sequencing were run (as pictured) for all samples except CPCG0004R, for which six lanes were sequenced.

FIG. 3 shows an overview of the library processing pipeline according to the present disclosure. A summary of the processing pipeline run for a single normal library. For each library, every lane-level BAM was first filtered individually. A set of all possible lane groupings was then generated consisting of all individual lanes, all pairs of lanes, all sets of three lanes, etc. (labelled as Group 1, Group 2, etc.). The lane-level BAMs in each grouping were merged and collapsed to produce group-level BAMs. These BAMs were then evaluated using the quality metrics listed in Table 2.

FIG. 4 shows the distribution of metric values for single-lane groupings according to the present disclosure. The metric values measured for all single-lane groupings showed varying distributions. The distribution of values between tissue types was also slightly different, with normal samples (n=38) showing generally lower values than tumour samples (n=60).

FIG. 5 shows coverage data for sequencing of two tumour libraries according to the present disclosure. Overall collapsed coverage for sample CPCG0001P (a) was much lower than CPCG0020P (b), which showed similar coverage to all other tumour data, indicating that CPCG0001P is a low-complexity library. For any given coverage depth, the percentage of individual bases reaching this depth was also lower in CPCG0001P (c) than CPCG0020P (d) and the other samples. Data points represent mean values across all groupings of the same size. Error bars represent standard error. Dashed lines represent 50× coverage.

FIG. 6 shows distribution of base-wise quality scores for two tumour libraries according to the present disclosure. The percentage of bases achieving a given minimum quality score was lower in CPCG0001P (a) than CPCG0020P (b) and the other samples. This indicates a smaller number of reads covering each base in the collapsed data, likely resulting from lower library complexity. In both samples the distribution does not change linearly with group size, indicating that information content eventually becomes saturated. Data points represent mean values across all groupings of the same size. Bars represent standard error. Dotted line represents the maximum quality score of 283 (calculated via SAMtools).

FIG. 7 shows pair-wise metric correlations across all groupings according to the present disclosure. To determine which metrics were highly related, Spearman's rank correlation coefficients (ρ) were calculated between every possible pair of metrics using the values of the metrics measured across all groupings. Correlations were calculated separately for normal data (a) and tumour data (b). In both tissue types, all metric pairs showed high correlations (ρ>0.70, p<10-70). Subtracting the correlations in tumour data from those in normal data (c) shows that in almost all cases, correlations are weaker in tumour (i.e. mostly positive values). Clustering was performed using the DIANA divisive hierarchical clustering algorithm.

FIG. 8 shows correlations between single-lane and all-lane values according to the present disclosure. To assess which metrics showed correlated single-lane and multi-lane values, Spearman's correlation (rho) was calculated for each between single-lane values and corresponding all-lane (4 for normal, 6 for tumour) values. Metrics are ordered by descending tumour correlation (left to right). Asterisk (*) represents metrics with significantly different correlation values (p<0.05) between tumour and normal data²⁷.

FIG. 9 are graphs showing tumour linear models for all metrics according to the present disclosure. For each metric, linear models were trained to predict two-lane values from single-lane data. (a) Each row represents a model trained to predict the given two-lane metric and each column represents a single-lane metric used as an input term in the model. Coefficients for each term are represented by dot size and colour, and p-values are indicated by the background colour with darker green representing lower p-values (asterisk (*) represents p=0.05). Black cells represent N/A values arising from single-lane metrics not being used to predict their own behaviour. No model was created for predicting number of clusters as this was only measured for raw single-lane groupings. All models were trained on 7 randomly selected tumour samples and validated on 3 independent tumour samples. Covariate bars summarize the accuracy of the models. (b) The model that predicts two-lane collapsed coverage shows a strong correlation between predicted and observed values (ρ=0.77; p<10⁻¹⁸) and a Lin's concordance correlation coefficient (CCC) of 0.59. Dashed lines represent +/−2× coverage. NB: the linear models in (a) were trained and tested using standardized metric data while the model in (b) used raw data (see Online Methods).

FIG. 10 shows predicted and observed coverage values for normal testing data according to the present disclosure. The linear model trained with seven tumour samples was moderately accurate at predicting two-lane collapsed coverage for nine normal samples, with a Spearman's rho of 0.8 (p<10-28) and Lin's concordance correlation coefficient (CCC) of 0.68. This indicates that the accuracy of the model is only partially dependent on the type of data used for training. Dashed lines represent +/−2× coverage.

FIG. 11 shows residuals of collapsed coverage predictions made by the tumour linear model according to the present disclosure. Residuals between predicted and observed collapsed coverage in the validation cohorts showed no trends with respect to single-lane collapsed coverage value in either (a) tumour testing data or (b) normal testing data. This indicates homoscedasticity (i.e. lack of systematic bias) in the data.

FIG. 12 shows the accuracy of preseq²³ complexity predictions. We compared preseq's predicted complexity curves with observed 6-lane data in our tumour samples. Solid black lines represent predicted complexity, and shaded region represent 95% confidence interval. Points represent observed 6-lane data. In (a), distinct reads are measured as the number of reads in the 6-lane BAM after collapsing with Picard (http://picard.sourceforge.net). In (b)-(f), distinct reads are measured as the number of unique start points in the collapsed 6-lane BAM (for consistency with the preseq definition). Red points represent the observed number of total reads in 6-lane uncollapsed data, whereas blue points represent the number of total reads that would be expected by multiplying the single-lane count by 6. Dashed red lines represent the complexity of the real 6-lane BAM as generated by preseq. Dashed black lines represent counts in the single-lane input BAM. (a) The preseq definition of uniqueness did not account for information gained from paired-end data, so the tool consistently underestimated the number of distinct reads in our data (CPCG0042P). (b) When the number of unique start points was considered instead, preseq showed much higher accuracy (CPCG0042P). (c) In cases where the single-lane input BAM had an unusual number of reads compared to other lanes in the sample, the estimated 6-lane read count would be highly divergent from the observed 6-lane count (CPCG0063P). (d-f) Testing input from different samples showed slightly varying accuracy, but in general the preseq tool produced a highly accurate estimate of library complexity (CPCG0007P, CPCG0183P, and CPCG0184P, respectively).

FIG. 13 shows that metric values show significant differences between the two coverage outcome classes. The heatmap shows the standardized metric values (z-scores) for each lane from the 15 tumour training samples. To determine if individual metrics could distinguish between the two outcome classes (<50× vs. ≧50× collapsed coverage), a Wilcoxon rank-sum test was performed between the lanes in each class. The p-values for these tests are summarized in the barplot to the right of the heatmap, with metrics sorted by p-value significance. In total, 12/15 metrics show significant differences between classes (p<10-3) and these metrics come from multiple categories, demonstrating the predictive value of a multi-metric model.

FIG. 14 shows that the principal components analysis (PCA) demonstrates that multiple metrics contribute to lane-level variance. PCA was performed using the metric data from all lanes in the 15 tumour training samples. (a) The proportion of variance explained (PVE) by each of the first 15 principal components (PCs) indicates that the first 2 PCs capture the majority of the lane-to-lane variation. The cumulative PVE of PC1 and PC2 is 84%. (b) Projecting the data onto PC1 and PC2 shows clear separation between lanes in the two outcome classes (<50× vs. ≧50× collapsed coverage), indicating that the first two PCs can distinguish between the categories. (c) The coefficient loadings for the first 3 principal components (PC1-PC3) are summarized. Dot size and colour represent coefficient values. The approximately uniform values for the loadings in PC1 indicate the importance of multiple metrics in explaining lane-to-lane variance.

FIG. 15 shows graphs containing data indicating that a random forest classifier according to the present disclosure accurately predicts coverage. A model was trained using 15 randomly selected tumour samples and validated on 12 independent tumour samples. (a) A ROC curve generated from the classifiers vote-number shows an AUC of 0.993. The black point represents the chosen default operating point corresponding to majority voting. (b) The variable importance for each metric (measured as the mean decrease in accuracy when that variable is omitted 36) demonstrates that many metrics influence classification accuracy. (c) The fraction of yes votes (confidence that 6 lanes will achieve 50× coverage) received by each single-lane input generally shows separation between true positive and true negative observations (black vs. grey bars). Covariates representing metric values for single-lane inputs show that multiple metrics influence the number of votes received.

FIG. 16 shows that a random forest trained with normal data generated at various time points showed poor classification ability. Training data came from 14 randomly selected normal samples generated at varying time points. The classifier was validated using data from the remaining 12 normal samples. (a) A ROC curve for the classifier shows a moderate area under the curve (AUC) of 0.618. Black point represents the chosen votes cut-off of 0.5. At this cut-off, the classifier had high accuracy and sensitivity values. However, the forest showed a very low specificity value. (b) The variable importance for each metric (measured as the mean decrease in accuracy when the variable is omitted) demonstrates that a variety of metric types are influential in classifying the data as achieving or not achieving 30× coverage. This forest consisted of 100,000 trees. (c) The fraction of yes votes (confidence that 4 lanes will achieve 30× coverage) received by each single-lane input shows no obvious separation between true positive and true negative observations (black vs. grey bars), indicating a lack of sufficient training information. Covariates represent metric values for single-lane inputs.

FIG. 17 shows that a tumour random forest trained with a subset of input lanes showed reduced performance. The forest was trained using only 4 of the 6 lanes from each training sample as input, and was tested on all lanes from the 12 validation tumours. The forest had a reduced accuracy of 88.9% in the validation cohort, and the fraction of yes-votes received by the single-lane testing inputs showed data from both outcome classes being incorrectly assigned.

FIG. 18 demonstrates that lanes from the same sample received a similar fraction of yes votes. For the 12 tumour validation samples, every single-lane grouping was assigned a fraction of yes votes by the random forest. The absolute difference in the votes was calculated between every pair of lanes. Lanes from the same sample (yellow) received a more similar fraction of yes votes as compared to lanes from different samples (green).

FIG. 19 demonstrates that the total number of clusters is variable between tumour validation samples. Individual lanes are represented by points, and samples are sorted by ascending intra-sample variance. Most samples showed a fairly uniform cluster count across lanes, but CPCG0063P and CPCG0103P (shaded background) contained some lanes with fewer clusters. This likely represents inconsistency in the amount of input DNA.

FIG. 20 shows that tumour sample cellularity influences the number of yes votes assigned by the random forest. The fraction of yes votes received by lanes in the tumour validation set shows a positive relationship with sample cellularity. A simple linear regression model fit to the data (dashed line) showed a significant slope of 1.7 (p=2.16×10−8; y-intercept=-0.65). The Spearman's rank correlation coefficient between fraction of yes votes and sample cellularity had a positive value of 0.36 (p=1.26×10−3), indicating a weak positive correlation between the two.

FIG. 21 shows that differences in run date cause reduced prediction accuracy. A random forest classifier trained with the 10 oldest tumour samples showed moderate prediction accuracy when used to classify the newest 17 tumour samples. (a) A receiver-operating characteristic curve showed a strong area under the curve (AUC) value of 0.741, but this is a reduction when compared to the classifier trained with non-time-separated data (FIG. 3). (b) The fraction of yes votes assigned to the samples by the random forest showed no clear separation between true positive and true negative observations (black vs. grey bars), indicating that the classifier is not sufficiently trained to recognize the two outcome types.

FIG. 22 shows that classification from lower-depth sequencing yields high predictive accuracy according to the present disclosure. ROC curves for random forests trained with half-lane, quarter-lane and eighth-lane data show high predictive accuracy on independent validation data. The AUC value of 0.993 when using full-lane data is only modestly reduced by reducing the amount of sequencing data used for classification (e.g. AUC of 0.967 for eighth-lane). Dots represent the operating point of majority-classification.

FIG. 23 shows the SeqControl workflow for metric collection and classifier training. In this example, the forest is designed to use a single lane to predict whether 6 lanes of sequencing will achieve 50× collapsed coverage. (a) For each training sample, BAMs representing all 6 individual lanes plus the pooled 6-lane BAM are run through the SeqControl scripts. This outputs a tab-delimited file summarizing metric values for each of the input BAMs (including the pooled BAM). (b) A tab-delimited file is created for classifier training where each row represents an observation. Single-lane metric values are used as input variables, and the true outcome class (whether 6 lanes reach 50× coverage) is determined using the pooled data. Data from all training samples are added to the training file. Note that this file must contain a column named “output”, and all other columns are used as features. (c) The file is passed to the fit_random_forest.R script, where a random forest classifier is trained. The trained forest object is saved for later use.

FIG. 24 shows the SeqControl workflow for classifier testing with a validation set. (a) For each testing sample, one or more newly sequenced lanes are run through SeqControl scripts to generate metric data. (b) A tab-delimited file is created for testing, where each row consists of single-lane metric values for input to the classifier. Data from multiple testing samples can be included in the file, and a prediction will be generated for each row. Note that all columns (features) used in the random forest training data must also be present in the testing file. (c) The testing file and the trained random forest object are passed to apply_random_forest.R. This outputs an updated version of the testing file that contains the predicted class for each row—in this case, whether 6 lanes of sequencing for the library are predicted to yield 50× coverage. The fraction of trees in the forest that assigned a yes vote to the row is also included in the output.

FIG. 25 shows KKNN 10-fold Cross Validation on Blood Samples and Tumour Samples. A weighted k-nearest neighbours model was tuned, and revealed that using k=2 and a distance of 2 with a triangular kernel yielded the best model. The space was tuned over of k from 1 to 100 and distance from 1 to 100. Ten fold cross validation was performed on the tuned model to create the ROC curves provided. This was an analysis of 508 lanes of normal and 843 lanes of tumour using the full set of expanded metrics.

### DETAILED DESCRIPTION

Since NGS studies suffer from biases introduced during library preparation¹⁷ and sequencing¹⁸, a key part of increased automation is quality control to ensure outputs meet pre-specified constraints. For instance, it is critical to pre-determine the amount of sequencing required to achieve a specific outcome (e.g. some minimum statistical confidence in identifying variants). Over-sequencing incurs time and money costs, while under-sequencing causes reduced prediction accuracy or delays for additional data collection. As another example, groups developing and evaluating new protocols must rapidly assess if data quality is improved or degraded.

To date, such techniques have been elusive, with only a handful of heuristic studies in the literature¹⁹⁻²³. Several factors confound the prediction of sequencing quality including variability amongst machines and reagent batches, and the complexity of sequencing libraries²³. Similarly the integrity and quality of DNA varies widely: clinical specimens that have been formalin-fixed and paraffin-embedded (FFPE) typically yield degraded DNA that is challenging to sequence^{24,25}. With large-scale exome and whole-genome sequencing studies²⁶ increasing in prevalence, the need for robust quality control is growing. To tackle these challenges, we introduce SeqControl: a technique for predicting overall experimental qualities using a small amount of sequencing, enabling production-scale use of NGS quality- and process-control.

Preferred embodiments of methods, systems, and apparatus suitable for use in implementing the invention are described through reference to the drawings.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In an aspect, there is disclosed a method of determining the amount of sequencing required to achieve a target sequencing quality of a genetic sample to be sequenced, the method comprising: receiving the genetic sample; sequencing a portion of the genetic sample; generating from the sequencing a sequencing quality metric, said sequencing quality metric belonging to a category of sequencing quality metrics; determining the amount of sequencing of the genetic sample required to achieve the target sequencing quality by inputting the sequencing quality metric into a model trained with a plurality of reference sequencing quality metrics to predict sequencing quality.

In some embodiments, the category of sequencing quality metrics is selected from the group consisting of overall coverage, coverage distribution, base-wise coverage, base-wise quality, sequencing experimental information, read-level sequence-quality, read-level mapping-quality, and coverage of genomic repeats.

In an embodiment, the sequencing quality metric belonging to the overall coverage category is selected from the group consisting of uncollapsed coverage, collapsed coverage, masked coverage and clusters. For each of these a summary statistic can be generated such as, but not limited to, the mean, median, standard-deviation, first-quartile and third-quartile.

In another embodiment, the sequencing quality metric belonging to the coverage distribution category is selected from the group consisting of unique start points and average reads/starts.

In yet another embodiment, the sequencing quality metric belonging to the base-wise coverage category is selected from the group consisting of percentage of bases that reach 0× coverage, percentage of bases that reach 1× coverage, percentage of bases that reach 2× coverage, percentage of bases that reach 3× coverage, percentage of bases that reach 4× coverage, percentage of bases that reach 5× coverage, percentage of bases that reach 6× coverage, percentage of bases that reach 7× coverage, percentage of bases that reach 8× coverage, percentage of bases that reach 9× coverage, percentage of bases that reach 10× coverage, percentage of bases that reach 20× coverage, percentage of bases that reach 30× coverage, percentage of bases that reach 40× coverage, percentage of bases that reach 50× coverage percentage of bases that reach 75× coverage, percentage of bases that reach 100× coverage, percentage of bases that reach 150× coverage, percentage of bases that reach 200× coverage, percentage of bases that reach 250× coverage, percentage of bases that reach 500× coverage, and percentage of bases that reach 1000× coverage.

In other embodiments, the sequencing quality metric belonging to the base-wise quality category is selected from the group consisting of percentage of bases receiving a base-wise genotype quality score greater than 0, percentage of bases receiving a base-wise genotype quality score of at least 10, percentage of bases receiving a base-wise genotype quality score of at least 20, percentage of bases receiving a base-wise genotype quality score of at least 30, percentage of bases receiving a base-wise genotype quality score of at least 40, percentage of bases receiving a base-wise genotype quality score of at least 50, percentage of bases receiving a base-wise genotype quality score of at least 60, percentage of bases receiving a base-wise genotype quality score of at least 70, percentage of bases receiving a base-wise genotype quality score of at least 80, percentage of bases receiving a base-wise genotype quality score of at least 90, percentage of bases receiving a base-wise genotype quality score of at least 100, and percentage of bases receiving the maximum base-wise genotype quality score.

In yet another embodiment, the sequencing quality metric belonging to the sequencing experimental information category is selected from the group consisting of Machine ID, machine-name, cluster density, technician name or technician ID.

In another embodiment, the sequencing quality metric belonging to the read-level sequence-quality category is selected from the group consisting of average per-read quality, maximum per-read quality, median per-read quality, SD of per-read quality, first-quartile of per-read quality and third-quartile of per-read quality.

In yet another embodiment, the sequencing quality metric belonging to the read-level mapping-quality category is selected from the group consisting of mapping quality of the read, average mapping quality, median mapping quality, standard deviation of mapping quality, first quartile of mapping quality, third quartile of mapping quality.

In some embodiments, the methods disclosed herein generate a plurality of sequencing quality metrics. In an embodiment, the plurality of sequencing quality metrics consists of 2, 3, 4, 5, 6 , 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 sequencing quality metrics.

In yet another embodiment, the plurality of sequencing quality metrics comprises at least 2 sequencing quality metrics selected from 2 different categories of sequencing quality metrics.

In other embodiments, the plurality of sequencing quality metrics comprises at least 3 sequencing quality metrics selected from 3 different categories of sequencing quality metrics.

In yet other embodiments, the plurality of sequencing quality metrics comprises at least 4 sequencing quality metrics selected from 4 different categories of sequencing quality metrics.

In some embodiments, the model disclosed herein is trained by inputting the plurality of reference sequencing quality metrics into the model.

In some embodiments, the model comprises a random forest classifier, a neural network, K-nearest neighbours, support vector machines, linear regression, linear discriminant analysis, or decision trees.

In some embodiments, the portion of the genetic sample is greater than 0% and less than 100% of the genetic sample. In an embodiment, the portion of the genetic sample is less than 50%, less than 40%, less than 30%, less than 20%, less than 10%, less than 5%, or less than 3% of the genetic sample. In some embodiments, the portion of the genetic sample is at least 1% of the genetic sample. In yet other embodiments, the portion of the genetic sample is at least 2% of the genetic sample.

In another embodiment, the portion of the genetic sample is between 2% and 50% of the genetic sample.

In some embodiments, the genetic sample is a genome. In yet other embodiments, the genetic sample originates from a tumour genome.

In other embodiments, the genetic sample originates from a non-tumour genome. In yet other embodiments, the genetic sample is a targeted sequence of a portion of a genome. In one embodiment, the targeted sequence of a portion of the genome is an exome. In another embodiment, the targeted sequence of a portion of the genome is a targeted panel.

In some embodiments, the target sequencing quality is a sequencing depth. In one embodiment, the sequencing depth is between 1× and 500×. In another embodiment, the sequencing depth is between 10× and 100×. In yet another embodiment, the sequencing depth is greater than 1×. In another embodiment, the sequencing depth is 10×, 20×, 30×, 40×, 50×, 60×, 70×, 80×, 90×, 100×, 110×, 120×, 130×, 140×, 150×, 160×, 170×, 180×, 190× or 200×.

In another aspect, there is disclosed a system for genetic sequencing, the system comprising: a device for receiving a genetic sample; a device for sequencing one or more portions of the genetic sample; a device for capturing sequencing data; at least one processor configured to: generate signals for commencing sequencing the one or more portions of the genetic sample; during or after sequencing of the one or more portions, receiving sequencing data for the one or more portions; generating from the sequencing data, at least one sequencing quality metric, said at least one sequencing quality metric belonging to at least one category of sequencing quality metrics; and generating signals for continuing or aborting the sequencing of the same or additional portions of the genetic sample based on a determination of the amount of sequencing of the genetic sample required to achieve a target sequencing quality using said at least one sequencing quality metric and a model trained with reference sequencing quality metrics to predict sequencing quality.

In an embodiment, the at least one category of sequencing quality metrics is selected from the group consisting of overall coverage, coverage distribution, base-wise coverage, and base-wise quality.

In another embodiment, at least one sequencing quality metric belonging to the overall coverage category is selected from the group consisting of uncollapsed coverage, collapsed coverage, and masked coverage and clusters.

In still another embodiment, the at least one sequencing quality metric belonging to the coverage distribution category is selected from the group consisting of unique start points and average reads/starts.

In yet another embodiment, the at least one sequencing quality metric belonging to the base-wise coverage category is selected from the group consisting of percentage of bases that reach 0× coverage, percentage of bases that reach 1× coverage, percentage of bases that reach 2× coverage, percentage of bases that reach 3× coverage, percentage of bases that reach 4× coverage, percentage of bases that reach 5× coverage, percentage of bases that reach 6× coverage, percentage of bases that reach 7× coverage, percentage of bases that reach 8× coverage, percentage of bases that reach 9x coverage, percentage of bases that reach 10× coverage, percentage of bases that reach 20× coverage, percentage of bases that reach 30× coverage, percentage of bases that reach 40× coverage, percentage of bases that reach 50× coverage percentage of bases that reach 75× coverage, percentage of bases that reach 100× coverage, percentage of bases that reach 150× coverage, percentage of bases that reach 200× coverage, percentage of bases that reach 250× coverage, percentage of bases that reach 500× coverage, and percentage of bases that reach 1000× coverage.

In another embodiment, the at least one sequencing quality metric belonging to the base-wise quality category is selected from the group consisting of percentage of bases receiving a base-wise genotype quality score greater than 0, percentage of bases receiving a base-wise genotype quality score of at least 10, percentage of bases receiving a base-wise genotype quality score of at least 20, percentage of bases receiving a base-wise genotype quality score of at least 30, percentage of bases receiving a base-wise genotype quality score of at least 40, percentage of bases receiving a base-wise genotype quality score of at least 50, percentage of bases receiving a base-wise genotype quality score of at least 60, percentage of bases receiving a base-wise genotype quality score of at least 70, percentage of bases receiving a base-wise genotype quality score of at least 80, percentage of bases receiving a base-wise genotype quality score of at least 90, percentage of bases receiving a base-wise genotype quality score of at least 100, and percentage of bases receiving the maximum base-wise genotype quality score.

In some embodiments, the at least one sequencing quality metric comprises at least 2 sequencing quality metrics selected from 2 different categories of sequencing quality metrics.

In some embodiments, the at least one sequencing quality metric comprises at least 3 sequencing quality metrics selected from 3 different categories of sequencing quality metrics.

In some embodiments, the at least one sequencing quality metric comprises at least 4 sequencing quality metrics selected from 4 different categories of sequencing quality metrics.

In some embodiments, the model is trained by inputting the plurality of reference sequencing quality metrics into the model.

In one embodiment, the model comprises a random forest classifier, a neural network, K-nearest neighbours, support vector machines, linear regression, linear discriminant analysis, or decision trees.

In another aspect, there is disclosed a system for genome sequencing, the system comprising: a device for receiving a genome sample; a device for sequencing one or more portions of the genome sample; a device for capturing sequencing data; and at least one processor configured to perform the methods described herein.

FIG. 26 is a block schematic diagram of a system 2600 that may be provided for determining an amount of sequencing required to achieve a target sequencing quality of a genetic sample to be sequenced. The system 2600 is provided as an example, and there may be more, less, different, and/or alternate units. Computer-implemented components of the system 2600 may, for example, interoperate in performing various computer-implemented steps, such as receiving the genetic sample or data relating to thereof, conducting sequencing operations on the portion of the genetic sample, generating from the sequencing a sequencing quality metric and providing a determination of the amount of sequencing of the genetic sample required to achieve the target sequencing quality by inputting the sequencing quality metric into a model trained with a plurality of reference sequencing quality metrics to predict sequencing quality.

The system 2600 is a computer-implemented system, wherein aspects may be provided through computer implementation in the form of hardware, software, embedded software, firmware, etc., using various computing devices, such as servers, processors, memory, non-transitory computer-readable media, etc. In some embodiments, one or more networks 2670 may be utilized for electronic communication. The system 2600 may be provided as part of a single computing device (e.g., a supercomputer), or a series of distributed devices, some of which may be physical, and some of which may be virtual. For example, system 2600 may be provided in the form of distributed networking resources, such as a “cloud computing” implementation (for example, a distributed set of processors, storage, memory, etc., may co-operate in performing computational functions based on instructions from one or more controllers).

The system 2600 may be used to improve and/or streamline various aspects of healthcare and/or bioinformatics analytics, through for example, providing a determination identifying the amount of sequencing required to achieve a specific outcome. These types of analytics may require significant computational processing, and given the amount of resources and time involved in sequencing, such a reduction in sequencing may provide various advantages, while potentially avoiding overly reduced prediction accuracy, etc. The amount of sequencing required for a specific outcome may be influenced by various factors, such as the quality of the input genetic information, etc. The quality may be assessed through one or more quality metrics, etc., the quality being potentially determined through the conducting of a small amount of sequencing. The quality metrics may be assessed across various categories, and metrics may reflect coverage depth across the genome.

The system 2600 may include various computerized components that may be specifically configured for use in providing features related to genome sequencing, such as providing computerized determinations of the amount of sequencing required to achieve a target sequencing quality of a genetic sample, having regard to various characteristics related to a particular genome sample. Some computerized components may include or be connected to sensors and physical apparatuses adapted to perform various steps involved in sequence analysis, such as analyzing or extracting information from physical samples, searching against biological databases, applying various models, performing sequence alignment, identifying sequence differences, comparing sequences, etc.

In some embodiments, the system 2600 includes a genetic data receiver unit 2602, partial sequencer unit 2603, a quality assessment unit 2604, a training unit 2606, a threshold determination unit 2608 2608, a sequence prediction unit 2610 2610, a retraining unit 2612, and a DNA fragment library 2614.

The system 2600 may further include a data storage 2680, which may be used to store various aspects of electronic information, such as genetic information, quality metrics, prior determination threshold results, logical rules, etc.

In some embodiments, the system 2600 may further be adapted to interoperate with a sample receiver 2650. The sample receiver may be configured to receive a physical sample and to extract various biological (e.g., genetic) data from the sample. In some embodiments, the system may be configured to receive data from a data source (e.g., from a repository, provided sample information from a laboratory apparatus). In some embodiments, the system 2600 may further include a physical sample receiver. The genetic data receiver unit 2602 may be configured to receive genetic data from various sources. For example, the genetic sample may be a genome, may originate from a tumor genome, may originate from a non-tumor genome, may be a targeted sequence of a portion of a genome, may be a targeted sequence of a portion of the genome is an exome, or a targeted sequence of a portion of a genome (e.g., a targeted panel). The genetic sample may be derived from various types of tissue and tissue preparations, such as blood, frozen preparations, tumors, formaldehyde fixed paraffin embedded tissue, etc.

The partial sequencer unit 2603 may be utilized to conduct sequencing a portion of the genetic sample, generating from the sequencing various sequencing quality metrics. For example, a sequencing quality metric may be generated, belonging to a category of sequencing quality metrics. The quality metrics may be generated through the analysis of various correlations and correlation profiles, for example, determining relationships between different variables and factors.

The sequencing quality metric may be provided in various types of electronic representations, and in some embodiments, a plurality of sequencing quality metrics are generated. For example, a plurality of sequencing quality metrics may be generated, including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 sequencing quality metrics. In some embodiments, the plurality of sequencing quality metrics comprises at least 2 sequencing quality metrics selected from 2 different categories of sequencing quality metrics. In some embodiments, the plurality of sequencing quality metrics comprises at least 3 sequencing quality metrics selected from 3 different categories of sequencing quality metrics. In some embodiments, the plurality of sequencing quality metrics comprises at least 4 sequencing quality metrics selected from 4 different categories of sequencing quality metrics.

The sequencing quality metrics may, for example, be selected from the group consisting of overall coverage, coverage distribution, base-wise coverage, base-wise quality, sequencing experimental information, read-level sequence-quality, read-level mapping-quality, and coverage of genomic repeats, among others. For example, there may be metrics related to machine ID, machine-name, cluster density, technician name/ID, average per-read quality, maximum per-read quality, median per-read quality, SD of per-read quality, first-quartile of per-read quality, third-quartile of per-read quality, mapping quality of the read, average mapping quality, median mapping quality, SD of mapping quality, first quartile of mapping quality, third quartile of mapping quality, among others. The sequencing quality metric belonging to the overall coverage category may also be selected from the group consisting of uncollapsed coverage, collapsed coverage, masked coverage and clusters. For each of these, a summary statistic can be generated such as, but not limited to, the mean, median, standard-deviation, first-quartile and third-quartile. In some embodiments, a sequencing quality metric belonging to the coverage distribution category may also be selected from the group consisting of unique start points and average reads/starts.

In some embodiments, the sequencing quality metric belonging to the base-wise coverage category may be selected from the group consisting of percentage of bases that reach 0× coverage, percentage of bases that reach 1× coverage, percentage of bases that reach 2× coverage, percentage of bases that reach 3× coverage, percentage of bases that reach 4× coverage, percentage of bases that reach 5× coverage, percentage of bases that reach 6× coverage, percentage of bases that reach 7× coverage, percentage of bases that reach 8× coverage, percentage of bases that reach 9× coverage, percentage of bases that reach 10× coverage, percentage of bases that reach 20× coverage, percentage of bases that reach 30× coverage, percentage of bases that reach 40× coverage, percentage of bases that reach 50× coverage percentage of bases that reach 75× coverage, percentage of bases that reach 100× coverage, percentage of bases that reach 150× coverage, percentage of bases that reach 200× coverage, percentage of bases that reach 250× coverage, percentage of bases that reach 500× coverage, and percentage of bases that reach 1000× coverage, among others.

In some embodiments, the sequencing quality metric belonging to the base-wise quality category may be selected from the group consisting of percentage of bases receiving a base-wise genotype quality score greater than 0, percentage of bases receiving a base-wise genotype quality score of at least 10, percentage of bases receiving a base-wise genotype quality score of at least 20, percentage of bases receiving a base-wise genotype quality score of at least 30, percentage of bases receiving a base-wise genotype quality score of at least 40, percentage of bases receiving a base-wise genotype quality score of at least 50, percentage of bases receiving a base-wise genotype quality score of at least 60, percentage of bases receiving a base-wise genotype quality score of at least 70, percentage of bases receiving a base-wise genotype quality score of at least 80, percentage of bases receiving a base-wise genotype quality score of at least 90, percentage of bases receiving a base-wise genotype quality score of at least 100, and percentage of bases receiving the maximum base-wise genotype quality score.

The model application unit 2604 may be configured to apply one or more models, utilizing various quality metrics in performing an analysis. In some embodiments, the model application unit 2604 may be configured with a pre-defined target sequencing quality. In some embodiments, the model application unit 2604 may be configured to determine a target sequencing quality based on a particular desired outcome.

The model application unit 2604 may be configured to receive the one or more sequencing quality metrics into a model trained with a plurality of reference sequencing quality metrics to predict sequencing quality. In some embodiments, the genetic information may be analyzed through the use of various machine learning classification tools, binary models, etc. For example, the models may be configured such that particular metrics are weighted differently than others (e.g., based on importance).

For example, a model may be utilized that comprises one or a combination of a random forest classifier, a neural network, K-nearest neighbors, support vector machines, linear regression, linear discriminant analysis, or decision trees.

A training unit 2606 may also be configured to train the model applied by the model application unit 2604 by inputting the plurality of reference sequencing quality metrics into the model. The model may be refined (e.g., retrained) over time, for example, by validating the prediction ability of various models. For example, a random forest can be trained by the training unit 2606.

Based on the output of the model application unit 2604, the threshold determination unit 2608 may be configured to conduct a determination of the amount of sequencing of the genetic sample required to achieve the target sequencing quality.

For example, a threshold may be provided, wherein the threshold is a percentage value indicating that the portion of the genetic sample required is greater than 0% and less than 100% of the genetic sample. In some embodiments, the portion of the genetic sample required is less than 50%, less than 40%, less than 30%, less than 20%, less than 10%, less than 5%, or less than 3% of the genetic sample. In some embodiments, the portion of the genetic sample required is at least 1% of the genetic sample. In some embodiments, the portion of the genetic sample required is at least 2% of the genetic sample. In some embodiments, the portion of the genetic sample required is between 2% and 50% of the genetic sample.

The threshold may also be a sequencing depth. For example, a sequencing depth may be between 1× and 500×, between 10× and 100×, or greater than 1×, according to various embodiments.

The sequence prediction unit 2610 may be configured to provide control command instructions, for example, issuing control command instructions to start, and/or stop sequencing, based for example, on sequencing of a particular threshold being achieved based on the outputs of the threshold determination unit 2608, etc.

In some embodiments, the system 2600 may be implemented as an input into a genome sequencing unit 2654, and the system 2600, through the sequence prediction unit 2610, may be configured to provide various instructions as to when enough sequencing has been completed to achieve a target sequencing quality of a genetic sample to be sequenced.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

For simplicity only one computing device is shown but system 2600 may include more computing devices operable by users to access remote network resources and exchange data. The computing devices may be the same or different types of devices. The computing device 2600 includes at least one processor, a data storage device (including volatile memory or non-volatile memory or other data storage elements or a combination thereof), and at least one communication interface. The computing device components may be connected in various ways including directly coupled, indirectly coupled via a network, and distributed over a wide geographic area and connected via a network (which may be referred to as “cloud computing”).

For example, and without limitation, the computing device may be a server, network appliance, set-top box, embedded device, computer expansion module, personal computer, laptop, personal data assistant, cellular telephone, smartphone device, UMPC tablet, video display terminal, gaming console, electronic reading device, and wireless hypermedia device or any other computing device capable of being configured to carry out the methods described herein.

FIG. 27 is a schematic diagram of computing device, exemplary of an embodiment of system 2600. As depicted, computing device includes at least one processor 2702, memory 2704, at least one I/O interface 2706, and at least one network interface 2708.

Each processor 2702 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 2704 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 2706 enables computing device 27 to interconnect with one or more input devices, such as a keyboard, mouse, camera, touch screen and a microphone, or with one or more output devices such as a display screen and a speaker.

Each network interface 2708 enables computing device 27 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps

As can be understood, the examples described above and below, and illustrated herein are intended to be exemplary only.

### EXAMPLES

### Experimental Design

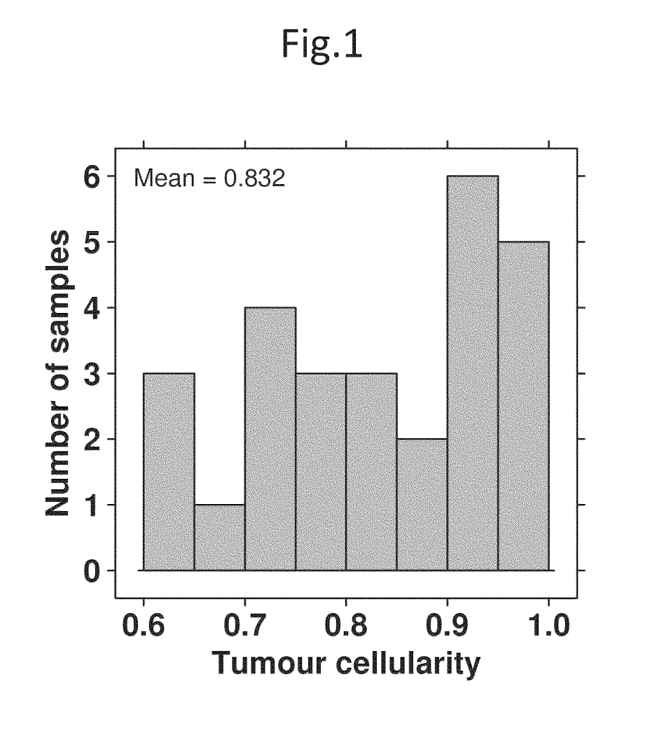

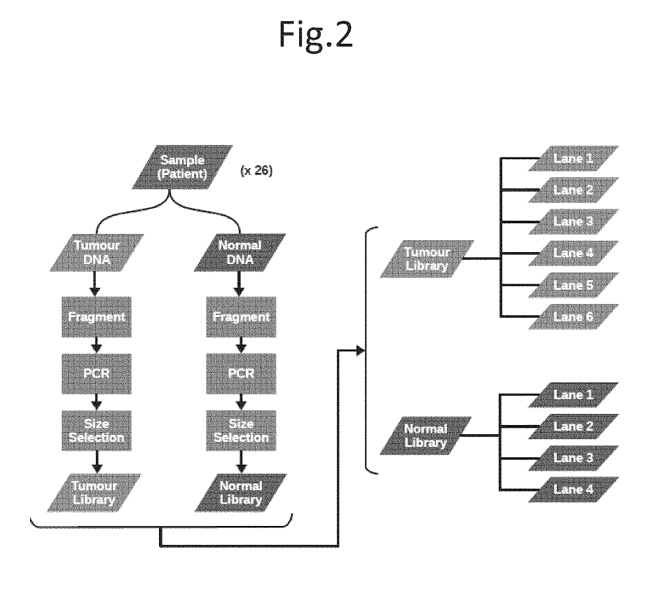

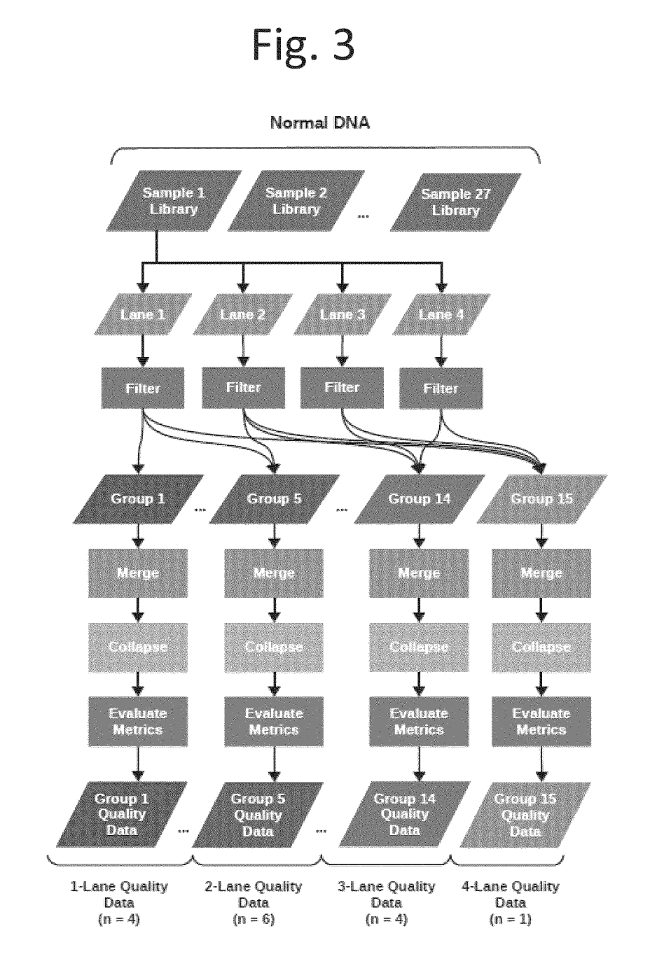

We studied 53 whole-genomes (26 blood-derived normals and 27 prostate tumours) sequenced as part of an International Cancer Genome Consortium²⁶ (ICGC) project studying intermediate-risk prostate cancer. Tumours were treatment-naïve and on average contained 83% tumour cells (Table 1; FIG. 1). A single library was created for each sample and sequenced on either six lanes (all tumours and one normal) or four lanes (remaining normals) of an Illumina HiSeq 2000 with a target of 50× coverage for tumours and 30× for normals (FIG. 2).

TABLE 1
Information about the CPC-GENE sample data.

| | | | | | Number of groupings | | | | | |
|---|---|---|---|---|---|---|---|---|---|---|
| | Tissue | Tissue | Tumour | Lanes | 1- | 2- | 3- | 4- | 5- | 6- |
| Sample name | type | preparation | cellularity²⁶ | sequence^{d} | lane | lane | lane | lane | lane | lane |
| CPCG0003R | Normal | Blood | N/A | 4 | 4 | 6 | 4 | 1 | N/A | N/A |
| CPCG0004R | Normal | Blood | N/A | 6 | 6 | 15 | 20 | 15 | 6 | 1 |
| CPCG0005R | Normal | Blood | N/A | 4 | 4 | 6 | 4 | 1 | N/A | N/A |
| CPCG0006R | Normal | Blood | N/A | 4 | 4 | 6 | 4 | 1 | N/A | N/A |
| CPCG0007R | Normal | Blood | N/A | 4 | 4 | N/G | N/G | 1 | N/A | N/A |
| CPCG0015R | Normal | Blood | N/A | 4 | 4 | 6 | 4 | 1 | N/A | N/A |
| CPCG0019R | Normal | Blood | N/A | 4 | 4 | 6 | 4 | 1 | N/A | N/A |
| CPCG0020R | Normal | Blood | N/A | 4 | 4 | 6 | 4 | 1 | N/A | N/A |
| CPCG0027R | Normal | Blood | N/A | 4 | 4 | 6 | 4 | 1 | N/A | N/A |
| CPCG0040R | Normal | Blood | N/A | 4 | 4 | 6 | 4 | 1 | N/A | N/A |
| CPCG0042R | Normal | Blood | N/A | 4 | 4 | N/G | N/G | 1 | N/A | N/A |
| CPCG0047R | Normal | Blood | N/A | 4 | 4 | N/G | N/G | 1 | N/A | N/A |
| CPCG0057R | Normal | Blood | N/A | 4 | 4 | N/G | N/G | 1 | N/A | N/A |
| CPCG0063R | Normal | Blood | N/A | 4 | 4 | N/G | N/G | 1 | N/A | N/A |
| CPCG0075R | Normal | Blood | N/A | 4 | 4 | N/G | N/G | 1 | N/A | N/A |
| CPCG0078R | Normal | Blood | N/A | 4 | 4 | N/G | N/G | 1 | N/A | N/A |
| CPCG0081R | Normal | Blood | N/A | 4 | 4 | N/G | N/G | 1 | N/A | N/A |
| CPCG0082R | Normal | Blood | N/A | 4 | 4 | N/G | N/G | 1 | N/A | N/A |
| CPCG0095R | Normal | Blood | N/A | 4 | 4 | N/G | N/G | 1 | N/A | N/A |
| CPCG0098R | Normal | Blood | N/A | 4 | 4 | N/G | N/G | 1 | N/A | N/A |
| CPCG0100R | Normal | Blood | N/A | 4 | 4 | N/G | N/G | 1 | N/A | N/A |
| CPCG0102R | Normal | Blood | N/A | 4 | 4 | N/G | N/G | 1 | N/A | N/A |
| CPCG0103R | Normal | Blood | N/A | 4 | 4 | N/G | N/G | 1 | N/A | N/A |
| CPCG0123R | Normal | Blood | N/A | 4 | 4 | N/G | N/G | 1 | N/A | N/A |
| CPCG0183R | Normal | Blood | N/A | 4 | 4 | N/G | N/G | 1 | N/A | N/A |
| CPCG0184R | Normal | Blood | N/A | 4 | 4 | N/G | N/G | 1 | N/A | N/A |
| CPCG0001P | Tumour | Frozen | 0.933038 | 6 | 6 | 15 | 20 | 15 | 6 | 1 |
| CPCG0003P | Tumour | Frozen | 0.7562336 | 6 | 6 | 15 | 20 | 15 | 6 | 1 |
| CPCG0004P | Tumour | Frozen | 0.6207995 | 6 | 6 | 15 | 20 | 15 | 6 | 1 |
| CPCG0005P | Tumour | Frozen | 0.9658808 | 6 | 6 | 15 | 20 | 15 | 6 | 1 |
| CPCG0006P | Tumour | Frozen | 0.7205436 | 6 | 6 | 15 | 20 | 15 | 6 | 1 |
| CPCG0007P | Tumour | Frozen | 0.955798 | 6 | 6 | N/G | N/G | N/G | N/G | 1 |
| CPCG0015P | Tumour | Frozen | 0.7375341 | 6 | 6 | 15 | 20 | 15 | 6 | 1 |
| CPCG0019P | Tumour | Frozen | 0.8858269 | 6 | 6 | 15 | 20 | 15 | 6 | 1 |
| CPCG0020P | Tumour | Frozen | 0.9067711 | 6 | 6 | 15 | 20 | 15 | 6 | 1 |
| CPCG0027P | Tumour | Frozen | 0.9441341 | 6 | 6 | 15 | 20 | 15 | 6 | 1 |
| CPCG0040P | Tumour | Frozen | 0.7270128 | 6 | 6 | 15 | 20 | 15 | 6 | 1 |
| CPCG0042P | Tumour | Frozen | 0.8342148 | 6 | 6 | N/G | N/G | N/G | N/G | 1 |
| CPCG0047P | Tumour | Frozen | 0.8434133 | 6 | 6 | N/G | N/G | N/G | N/G | 1 |
| CPCG0057P | Tumour | Frozen | 0.9145626 | 6 | 6 | N/G | N/G | N/G | N/G | 1 |
| CPCG0063P | Tumour | Frozen | 0.8212367 | 6 | 6 | N/G | N/G | N/G | N/G | 1 |
| CPCG0075P | Tumour | Frozen | 0.7608982 | 6 | 6 | N/G | N/G | N/G | N/G | 1 |
| CPCG0078P | Tumour | Frozen | 0.9467224 | 6 | 6 | N/G | N/G | N/G | N/G | 1 |
| CPCG0081P | Tumour | Frozen | 0.931469 | 6 | 6 | N/G | N/G | N/G | N/G | 1 |
| CPCG0082P | Tumour | Frozen | 0.9712882 | 6 | 6 | N/G | N/G | N/G | N/G | 1 |
| CPCG0095P | Tumour | Frozen | 0.9762753 | 6 | 6 | N/G | N/G | N/G | N/G | 1 |
| CPCG0098P | Tumour | Frozen | 0.9587494 | 6 | 6 | N/G | N/G | N/G | N/G | 1 |
| CPCG0100P | Tumour | Frozen | 0.719226 | 6 | 6 | N/G | N/G | N/G | N/G | 1 |
| CPCG0102P | Tumour | FFPE | 0.6235563 | 6 | 6 | N/G | N/G | N/G | N/G | 1 |
| CPCG0103P | Tumour | FFPE | 0.628531 | 6 | 6 | N/G | N/G | N/G | N/G | 1 |
| CPCG0123P | Tumour | Frozen | 0.8880499 | 6 | 6 | N/G | N/G | N/G | N/G | 1 |
| CPCG0183P | Tumour | Frozen | 0.7866281 | 6 | 6 | N/G | N/G | N/G | N/G | 1 |
| CPCG0184P | Tumour | Frozen | 0.6932734 | 6 | 6 | N/G | N/G | N/G | N/G | 1 |

(FFPE = formalin-fixed paraffin-embedded, N/A = not applicable, N/G = not generated)

We measured 15 sequencing data quality metrics across four categories: overall coverage, coverage distribution, base-wise coverage and base-wise variant-calling confidence scores (Table 2). Overall coverage metrics reflect average coverage depth across the genome. Coverage-distribution metrics describe how evenly reads are distributed. Base-wise coverage metrics measure the proportion of bases that reach a given coverage—a measure of sequencing uniformity. Base-wise SAMtools quality scores quantify confidence in per-base genotype predictions. All metrics can be rapidly evaluated with open-source software (Table 3) source code is available.

TABLE 2
Quality metrics used to evaluate sample data. Each metric was evaluated on the BAM files to
assess the quality of data in the grouping. See Table 3 for details about software and parameters used.

| | | | Software/Resource | |
|---|---|---|---|---|
| Metric | Definition | Purpose | used | Category |
| Uncollapsed | Number of bases | Observe how overall | Lengths of reads in | Overall coverage |
| Coverage, | in all reads | coverage changes as | BAM files | |
| Collapsed | divided by total | the number of lanes | | |
| Coverage | number of bases | increases | | |
| | in the reference | By comparing | | |
| | genome (UCSC hg19) | uncollapsed and | | |
| | | collapsed, we can see | | |
| | | how much of the multi- | | |
| | | lane coverage is | | |
| | | redundant, giving an | | |
| | | estimate of library | | |
| | | complexity | | |
| Masked Coverage | Number of bases | Shows collapsed | UCSC hg19 FASTA | |
| | in all reads | coverage when | files, length of reads | |
| | divided by total | uninformative bases | in BAM files | |
| | number of known | are not considered | | |
| | (not “N”) in the | (since it is not | | |
| | reference genome | possible to map to | | |
| | (UCSC hg19) | these locations) | | |
| Clusters | Total number of | Estimates the total | SAMtools (v0.1.18) | |
| | fragments sequenced | amount of DNA | view function used on | |
| | (including unmapped, | sequenced (may vary | raw (unfiltered) lane- | |
| | multi-mapped) | slightly between lanes) | level BAM files | |
| Unique start points | Number of | Gives an estimate of | Start coordinates of | Coverage distribution |
| | distinct locations | coverage distribution | reads in BAM files | |
| | in the reference | Shows roughly how | | |
| | genome at which | many new regions | | |
| | a read begins | are sequenced as lanes | | |
| | | are added | | |
| Average reads/start | Total number of | Estimates how many | Start coordinates | |
| | reads divided by | reads are located at | reads in BAM files | |
| | total number of | the same position | | |
| | unique start points | A value close to 1 | | |
| | | indicates low | | |
| | | redundancy in the | | |
| | | data | | |
| Percentage of bases | Percentage of known | Shows how many | BEDtools (v2.11.2) | Base-wise coverage |
| that reach various | bases in the reference | additional bases reach | genomeCoverageBed | |
| coverage depths | genome (UCSC hg19) | a given target coverage | function used on | |
| (0x, 8x, 20x, 30x, | that have been | depth (eg. 50x) by | BAM files | |
| 50x) | sequenced by some | adding more lanes | | |
| | minimum number of reads | Error bars (standard | | |
| | (1, 8, 20, 30, 50) | error) are also helpful | | |
| | | in showing any | | |
| | | large variation between | | |
| | | groups of the same size | | |
| Percentage of bases | Percentage of known | Shows what percentage | SAMtools (v0.1.18) | Base-wise quality |
| receiving a given | bases in the reference genome | of bases can be | mpileup and bcftools | |
| base-wise genotype | (UCSC hg19) that received | confidently genotyped | view functions used | |
| quality score (0, 50, | some minimum genotype | (assigned A, C, G, T or | on BAM files | |
| 100, max) | quality score (0, 50, 100, max) | multiple variation is | | |
| | | seen) | | |
| | | Allows us to see how | | |
| | | base call confidence | | |
| | | increases as lanes are | | |
| | | added | | |
| | | Note: The quality | | |
| | | scores represent the | | |
| | | Phred-scaled probability | | |
| | | that the base called is | | |
| | | incorrect (as described | | |
| | | in the VCF format | | |
| | | specification). so higher | | |
| | | scores indicate higher | | |
| | | confidence calls | | |
| | | Note: SAMtools outputs | | |
| | | a maximum score of | | |
| | | 283 | | |

TABLE 3
Command line parameters used for calculating SeqControl metrics.
Any parameters not listed below used default values.

| Processing Step | Software Used | Function Used | Parameters Specified |
|---|---|---|---|
| Aligning reads to the | Novoalign (v2.07.14) | novoalign | d db_name: pathname of indexed |
| reference genome | | | reference sequence (UCSC hg19) created |
| | | | by novoindex |
| | | | f reads1 reads2: names of FASTQ files |
| | | | containing paired end reads |
| | | | r ALL 5: report up to 5 alignments per |
| | | | read |
| | | | R 0: consider alignments unique if they |
| | | | differ in mapping quality |
| | | | o SAM: output in SAM format |
| Filtering lane-level | SAMtools | view | b: output in BAM format |
| BAM files | (v0.1.18) | | F 4: skip unmapped fragments |
| | | | F 256: skip secondary alignments |
| | | | q 30: skip alignments with MAPQ value |
| | | | smaller than 30 |
| Merging lane-level | Picard (v1.41) | MergeSamFiles | I=in_bam I=in_bam2 ... : input BAM files |
| BAM files | | | O=out_bam: output BAM file |
| | | | TMP_DIR=tmp_dir: directory to store |
| | | | temporary files |
| Collasping group- | Picard (v1.41) | MarkDuplicates | I=in_bam: input BAM file |
| level BAM files | | | O=out_bam: ouput BAM file |
| | | | METRICS_FILE=metrics_file: location to |
| | | | write metrics file |
| | | | TMP_DIR=tmp_dir: directory to store |
| | | | temporary files |
| | | | ASSUME_SORTED=true: ignore the sort |
| | | | order in the header file |
| | | | VALIDATION_STRINGENCY=LENIEN |
| | | | T: validation stringency for the BAM files |
| | | | read |
| | | | REMOVE_DUPLICATES=true: do not |
| | | | write duplicates to the output file |
| Generating genome- | BEDTools | genomeCoverage | ibam in_bam: input BAM file |
| wide coverage | (v2.11.2) | Bed | g chr_counts: file containing the number |
| | | | of bases in each chromosome in the |
| | | | genome |
| Calculating genotype | SAMtools | mpileup | Q 0: minimum base quality of 0 for a base |
| quality scores (step 1) | (v0.1.18) | | (include all bases) |
| | | | A: use anomalous read pairs |
| | | | I: do not perform INDEL calling |
| | | | g: compute genotype likelihoods |
| | | | u: output uncompressed BCF (preferred |
| | | | for piping) |
| | | | f fasta_ref: indexed reference sequence |
| | | | FASTA file (UCSC hg19) |
| Calculating genotype | Edited version of | bcftools view | N: skip sites where the REF field is not |
| quality scores (step 2) | SAMtools (v0.1.18) | (input piped from | A/C/G/T |
| | | mpileup call in | c: SNP calling (likelihood based analyses) |
| | | step 1) | g: call genotypes at variant sites |
| Calculating number | SAMtools | view | c: print a count of the total alignments |
| of clusters in raw | (v0.1.18) | | instead of printing the alignments |
| BAMS | | | F 256: skip secondary alignments |
| | | | F 128: skip second reads in a pair |

Do Different Quality Metrics Have Independent Predictive Power?

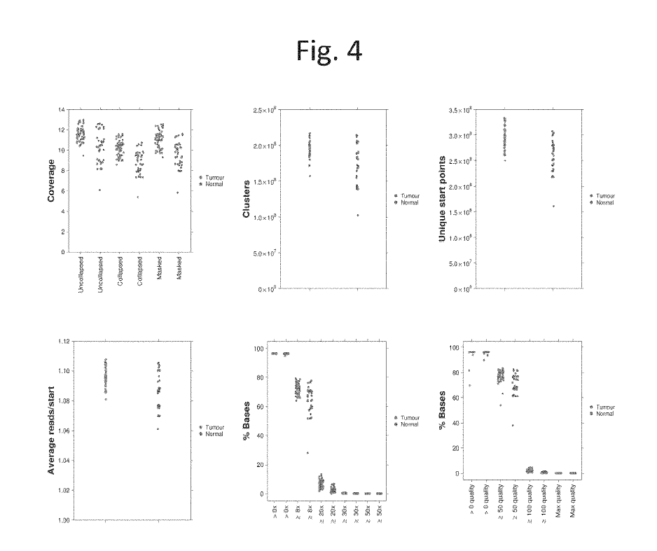

We first assessed the variability between different runs (i.e. lanes) of the same sequencing library to verify our dataset reflects the heterogeneity seen in large-scale sequencing datasets. For each sample we created all possible lane groupings: every individual lane, every pair of lanes, etc. (FIG. 3; Table 1). Data for each grouping was pre-processed and each quality metric was evaluated for every grouping (Tables 4 & 5, found in Appendix). We observed significant intra- and inter-sample variability, making this a realistic training dataset (FIGS. 4-6).

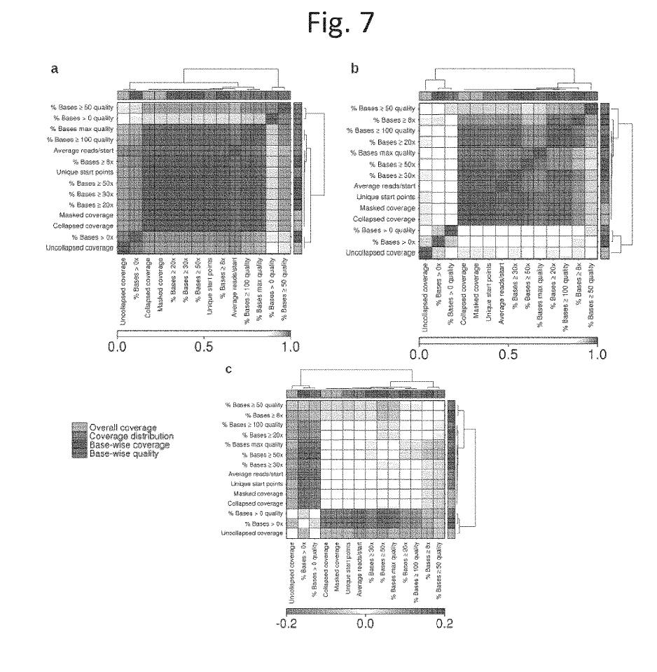

Next, we studied the relationship between each pair of metrics by calculating Spearman's rank correlation coefficient (p) separately for normal and tumour samples (FIG. 7a-b). Metrics were strongly, but imperfectly correlated (0.70<ρ<1; p<)10⁻⁷⁰). Intriguingly tumour and normal samples showed divergent correlation profiles, with most correlations being weaker in tumour-derived samples (FIG. 7c). This may reflect fundamental differences such as cellular heterogeneity within tumours. Thus, different quality metrics yield distinct but related information.

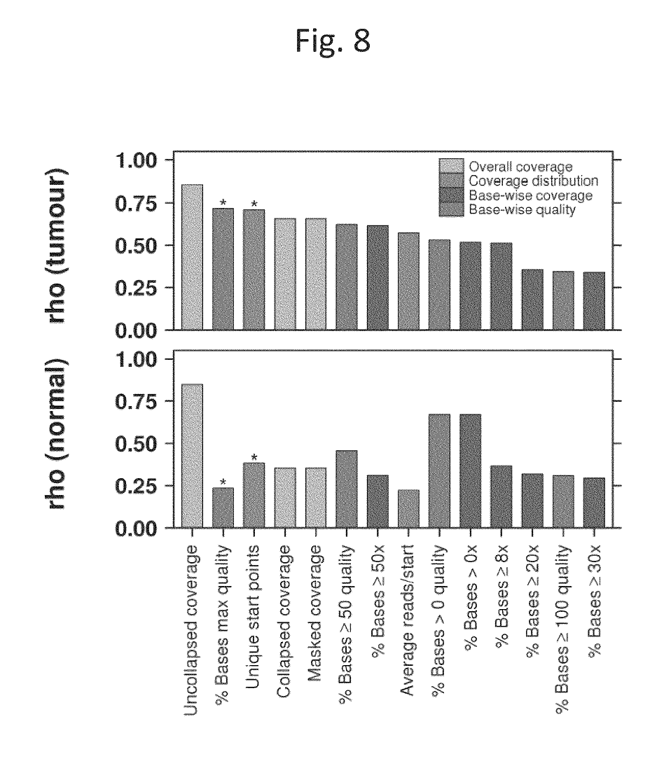

To exploit this result, we sought to determine if multi-lane behaviour could be predicted from single-lane values of a metric. We calculated correlations between single-lane values and all-lane values (4-lane for normal, 6-lane for tumour) for each metric (FIG. 8). Uncollapsed coverage showed a strong correlation (ρₙₒᵣₘₐₗ=0.85; ρₜᵤₘₒᵤᵣ=0.86), reflecting the similar amounts of total DNA sequenced in each lane. Surprisingly, no other metric showed a correlation greater than 0.75. In fact, some metrics showed very poor predictive ability, such as the percentage of bases with SAMtools quality scores above 100 (ρₙₒᵣₘₐₗ=0.31; ρₜᵤₘₒᵤᵣ=0.34). Tumour and normal samples were broadly similar, but differed significantly²⁷ for two metrics: the percentage of bases at maximum quality (ρₙₒᵣₘₐₗ=0.24; ρₜᵤₘₒᵤᵣ=0.72; p=0.003) and the number of unique start points (ρₙₒᵣₘₐₗ=0.38; ρₜᵤₘₒᵤᵣ=0.71; p=0.029).

Can Different Quality Metrics Predict One Another?

The strong pair-wise correlations between metrics (FIG. 7) and their moderate predictive power when used individually (FIG. 8) suggested models could be created to relate different quality metrics. We fit a linear model to predict each metric's two-lane value from single-lane data, modelling tumour and normal data separately. We randomly chose 7 samples for training and 3 for testing, and considered all possible pairs of lanes, yielding 210 training and 90 testing tumour observations (Table 1).

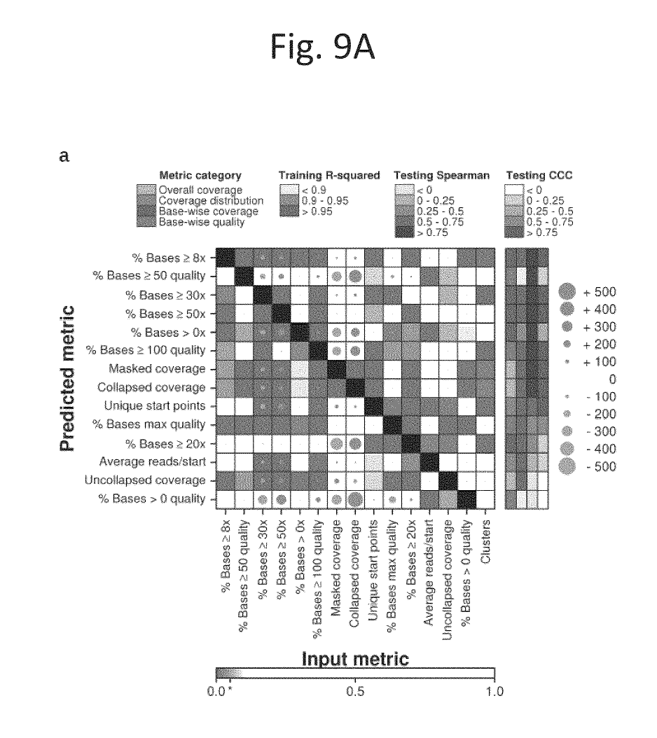

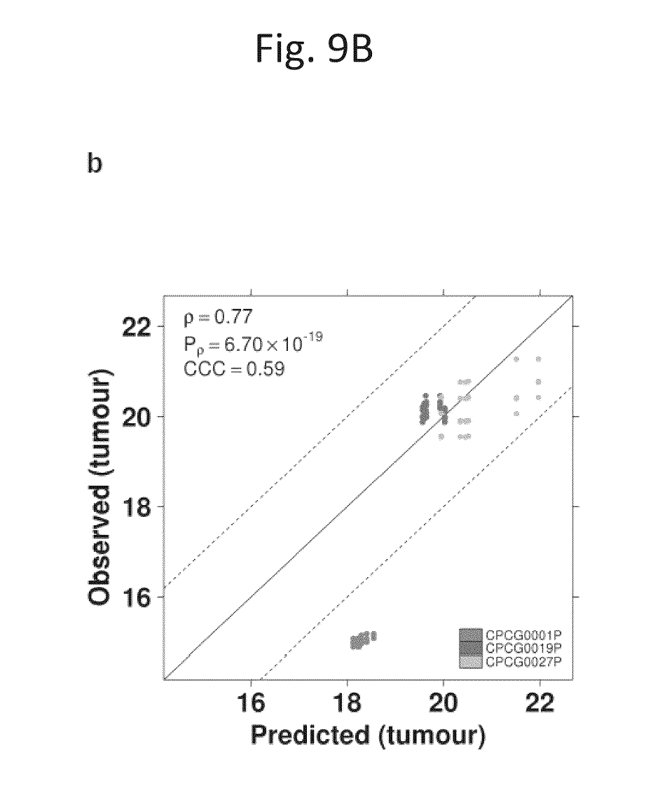

FIG. 9a summarizes the entire set of models for tumour data: each row represents a model trained to predict a two-lane metric value and each column represents a single-lane metric used as input. Most models showed strong predictive accuracy, with 7/14 achieving Lin's concordance correlation coefficient (CCC) values above 0.75. Interestingly, metrics that poorly account for redundancy (e.g. uncollapsed coverage) were harder to model than metrics that better reflect the complex relationship between quality and amount of sequencing (e.g. percentage of bases reaching 8×, ρ=0.94 and CCC=0.85).

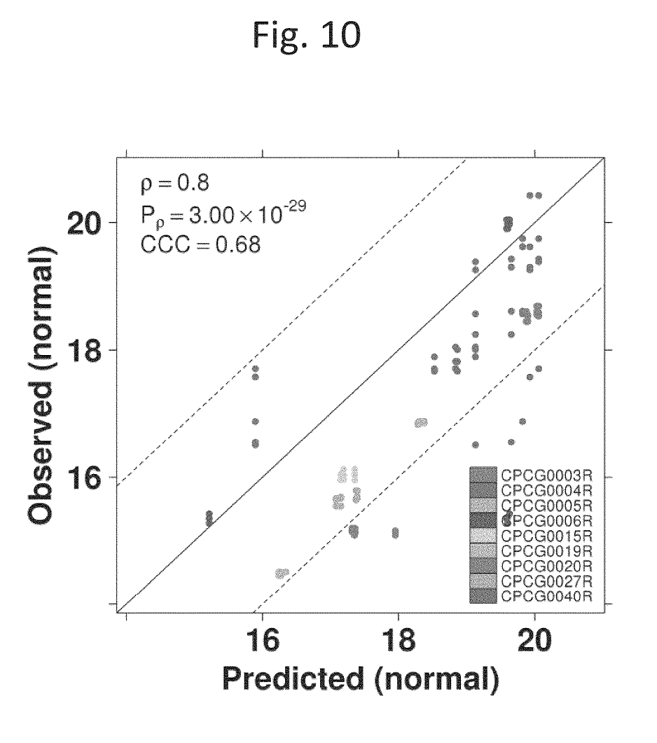

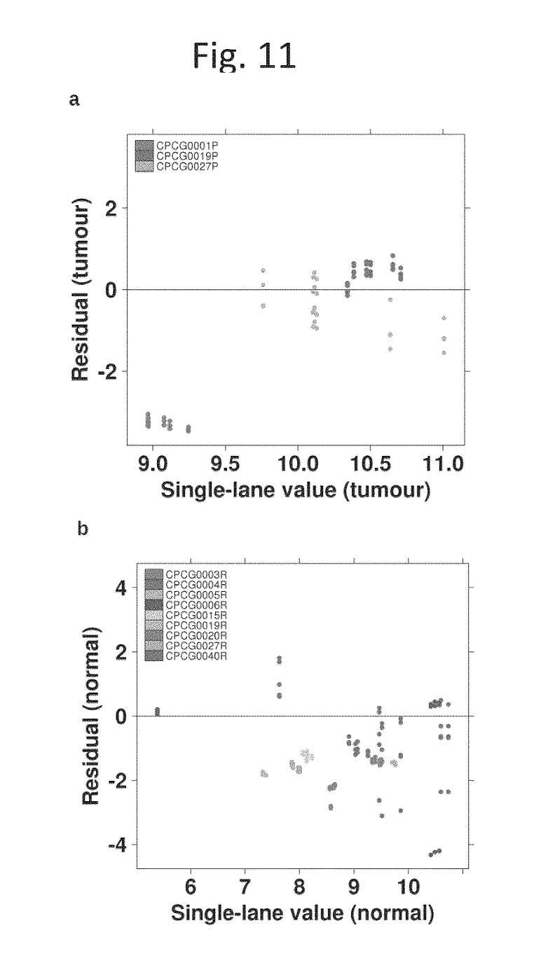

Since collapsed coverage is currently the most widely used target quality metric in sequencing centres, we further investigated this model. In the validation cohort (FIG. 9b) there was a strong correlation between predicted and observed coverage values (ρ=0.77; CCC=0.59). Further, the tumour model accurately predicted coverage for normal DNA samples (ρ=0.80; p=3.00×10⁻²⁹; FIG. 10), suggesting multi-metric models are less sensitive to library type. Residuals between observed and expected values were uncorrelated with single-lane coverage (FIG. 11), suggesting no systematic bias.

Can Production-Ready Models Be Developed?

While these linear models provide insight, production applications require rapid, reliable and accurate predictions. Binary predictions with confidence metrics are optimal for automation, suggesting the use of machine learning classification techniques. To demonstrate this approach, we started with a practical question. Most sequencing studies set a target coverage depth, but how many lanes of sequencing will be required to reach it for a given library?

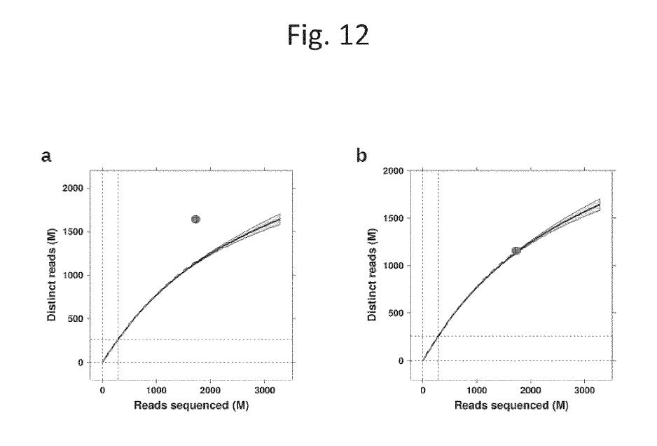

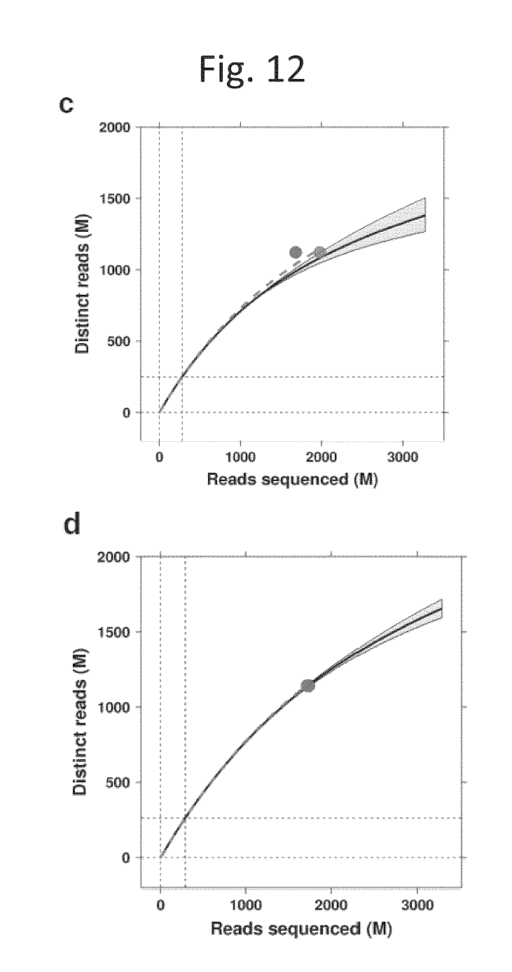

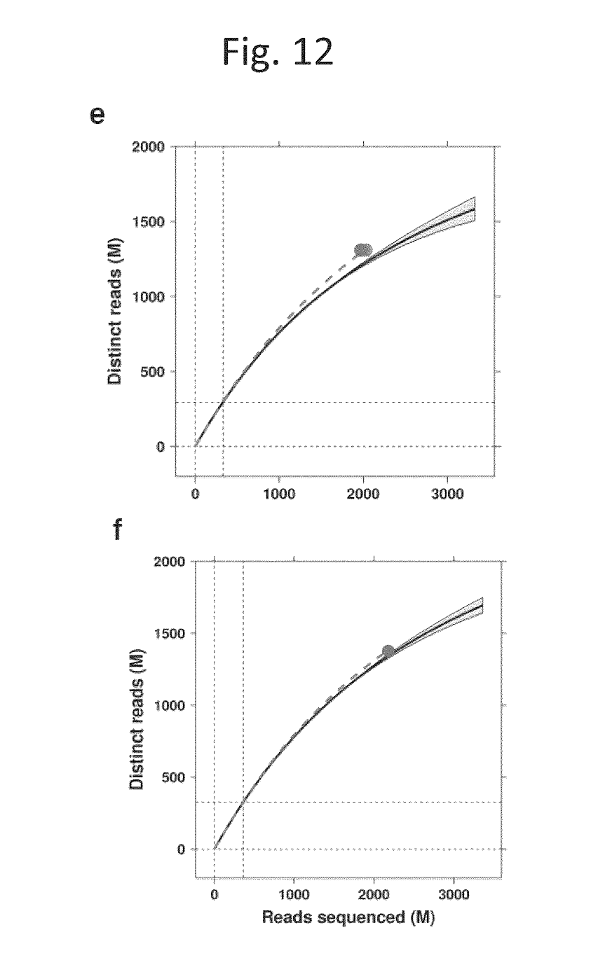

We began by assessing the performance of the state-of-the-art tool for predicting library complexity, preseq²³. Overall preseq failed to run for 52% (85/162) of our tumour sample lanes, including all FFPE data. Of the 48% (77 lanes) where results were generated, only 36 (47%) yielded preseq-estimated 95% confidence intervals that contained the true value (FIG. 12). Critically, FFPE samples (which represent routine clinical processing and exhibit degraded DNA with unusual mutational profiles^{24,25}) were all failed to run—exactly the real-world cases where a quality-control model is most needed. Thus preseq only produced reliable estimates for 22.2% (36/162) of lanes, highlighting an urgent need for improved predictions.

We therefore sought to directly predict if a given amount of sequencing would reach a pre-specified threshold. We used 15 randomly selected samples for training and the remaining 12 for validation. Ten of the validation samples were sequence from fresh-frozen tumour tissue while the remaining two used FFPE-preserved tissue. We selected our thresholds to reflect the most common targets in ICGC projects²⁶: for normal data, whether 4 lanes of sequencing would achieve 30× coverage; for tumour data, whether 6 lanes would achieve 50× coverage. Our classifier uses 15 metrics on a single lane to predict the results of multi-lane sequencing. Each validation lane was used, yielding 72 predictions for tumours and 48 for normals.

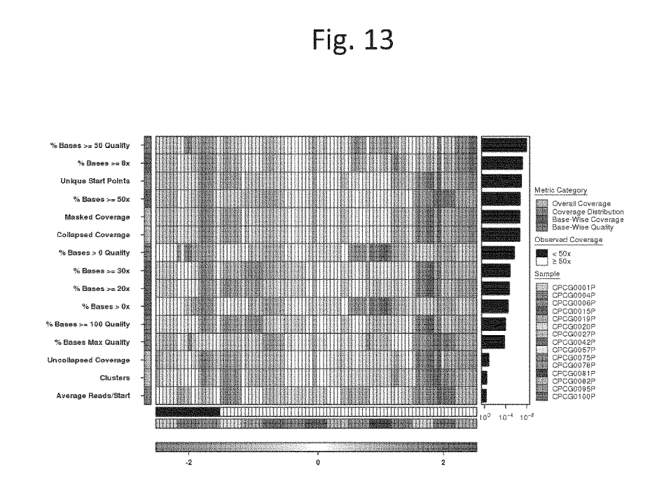

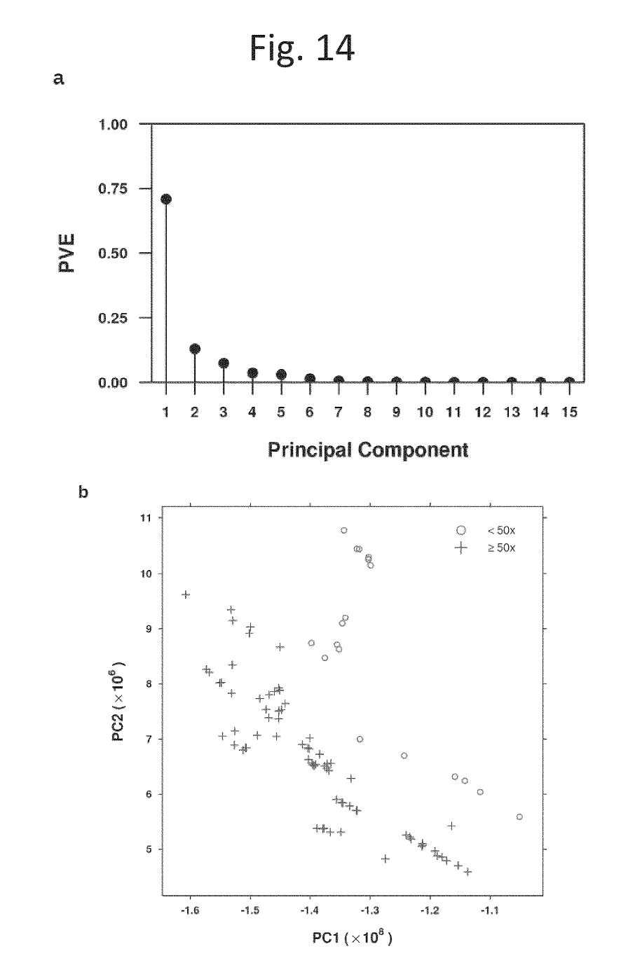

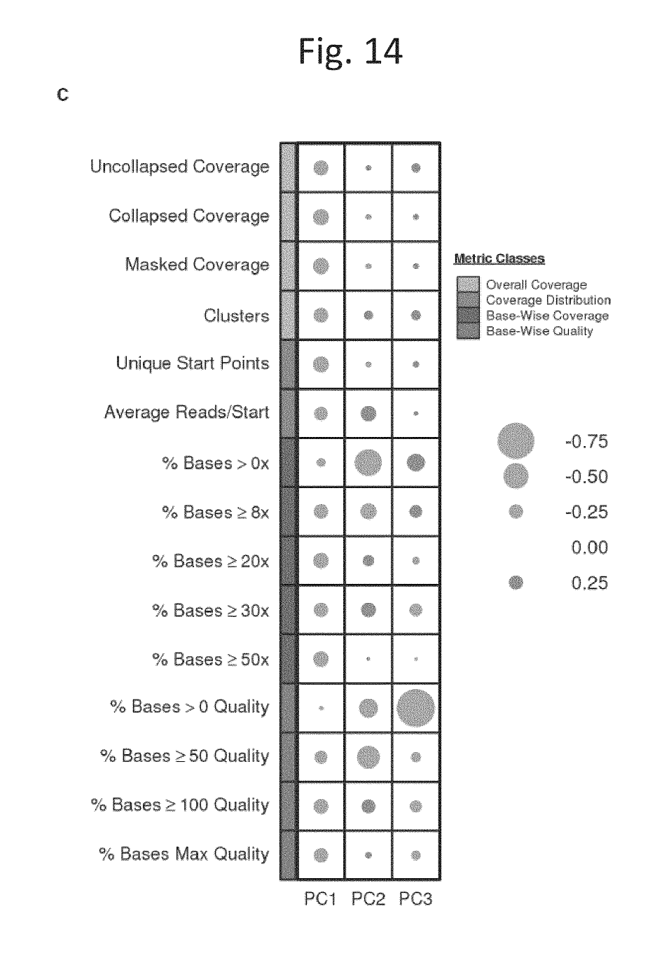

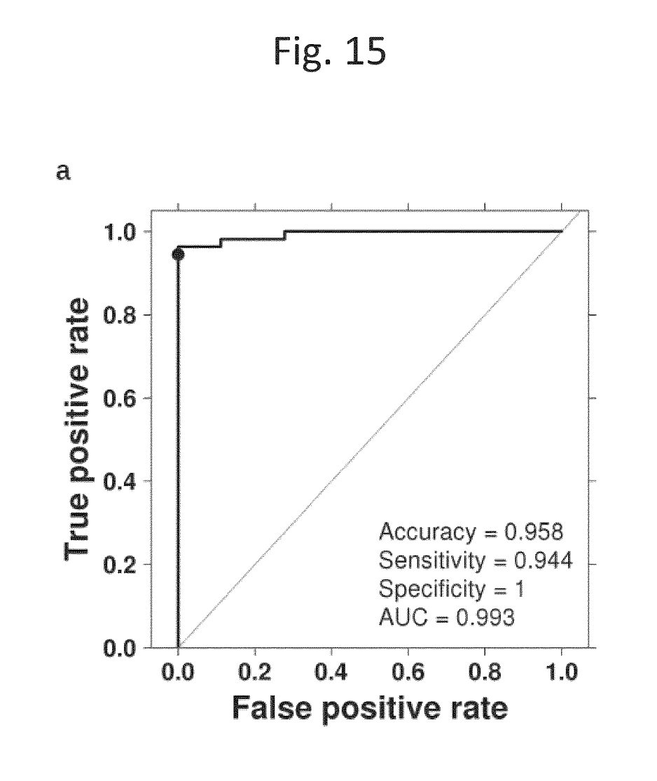

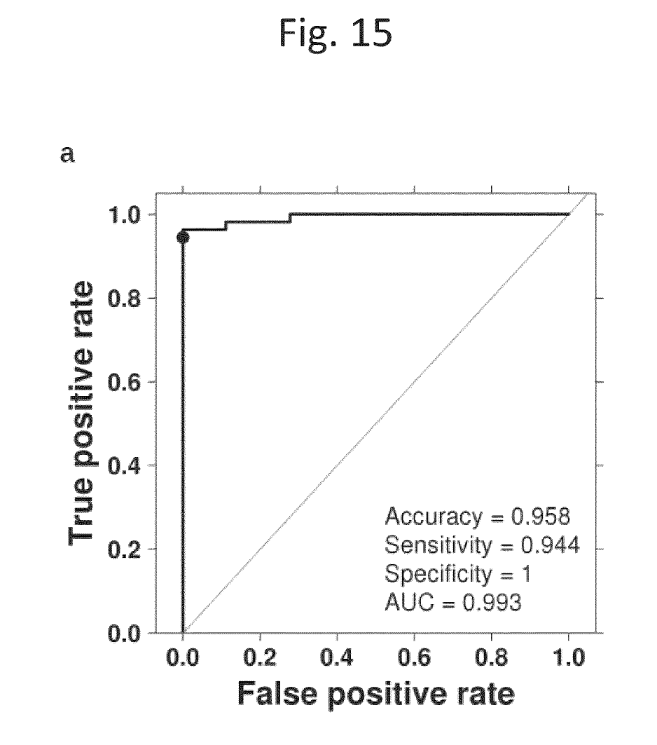

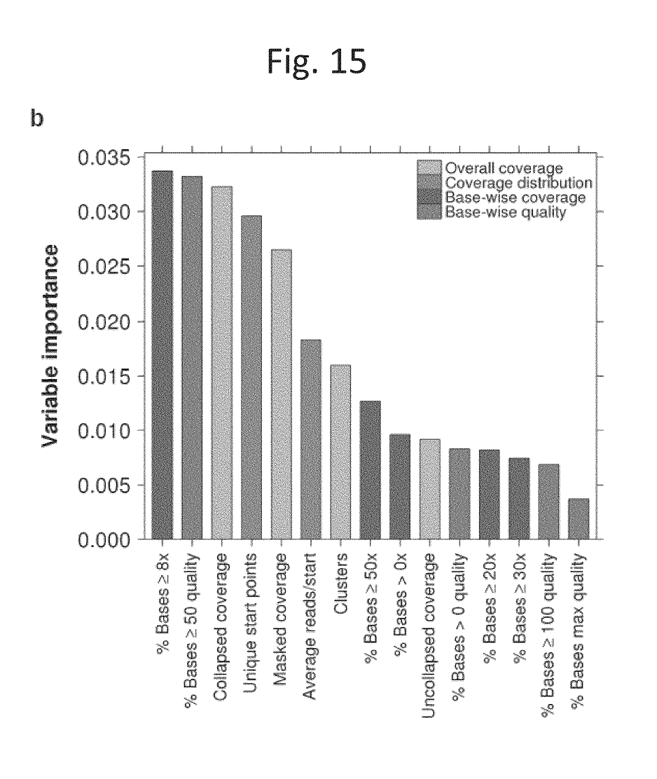

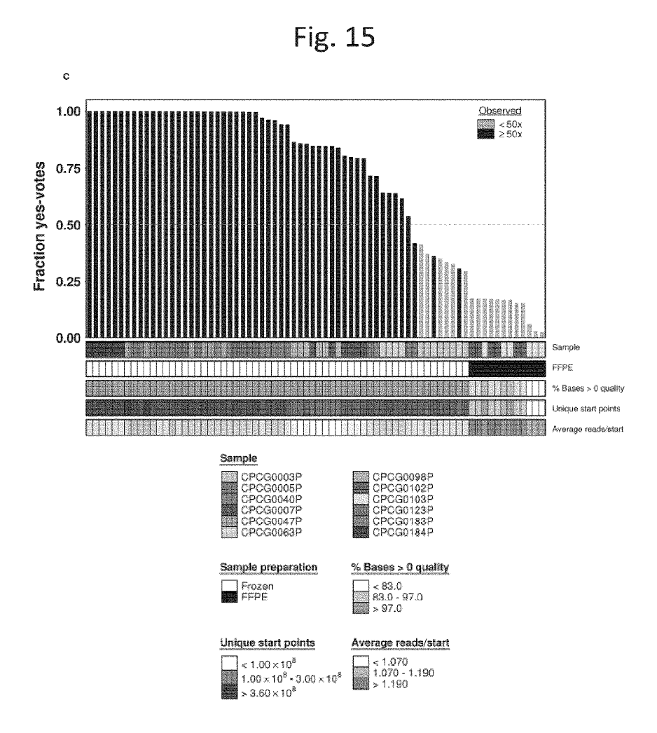

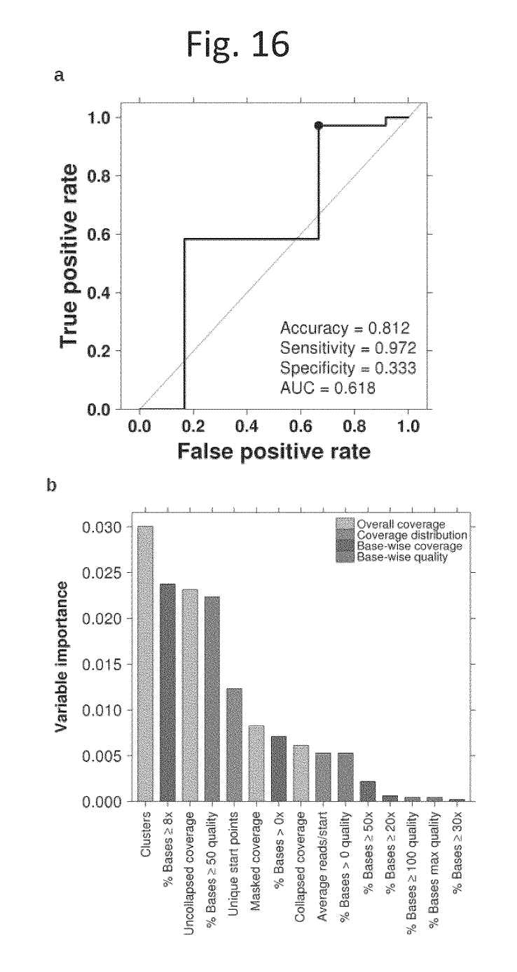

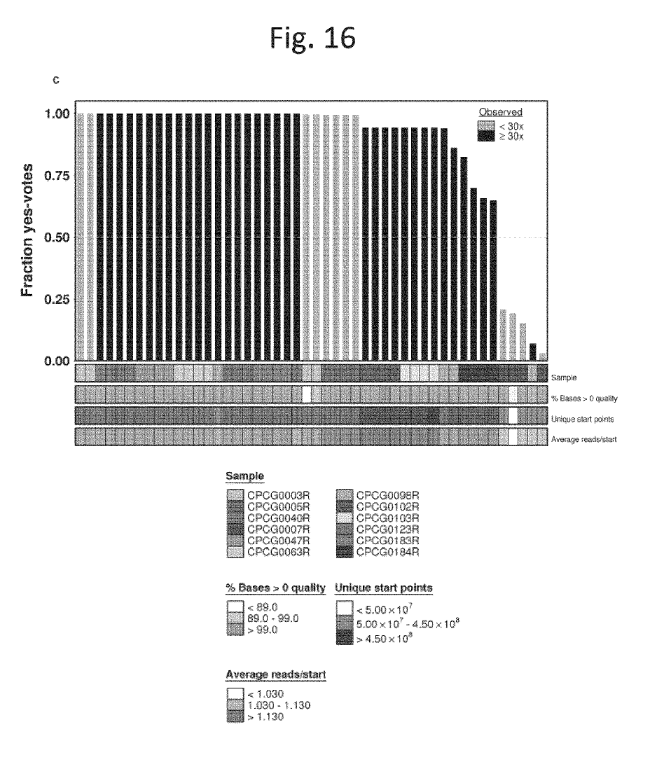

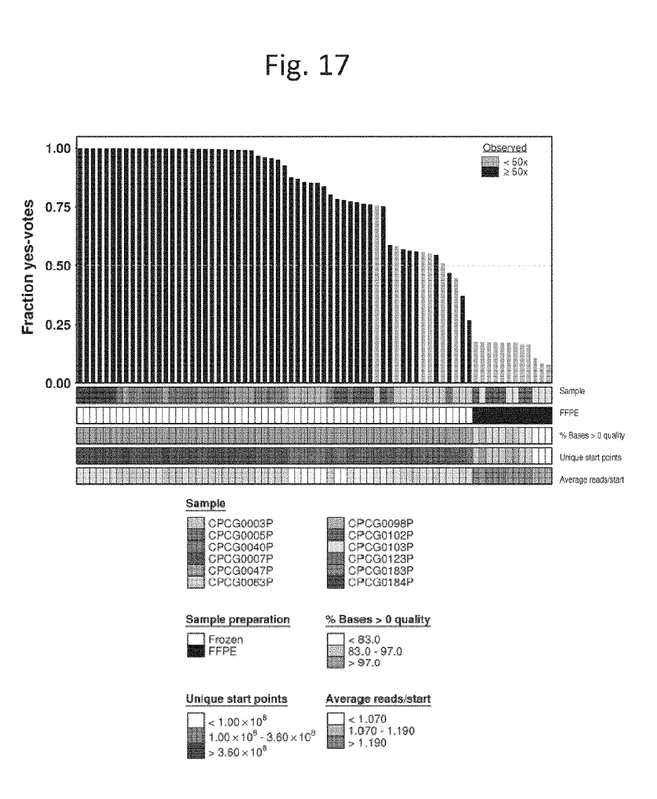

We began by testing if metrics univariately separated these two classes, and 12/15 did (Wilcoxon rank-sum test; p<10⁻³; FIG. 13). To test if the full set of metrics should be used, we performed principal components analysis and showed that each metric was important in the first three principal components (FIG. 14). We therefore trained separate random forest classifiers²⁸ for tumour and normal data. The tumour-prediction model had 95.8% accuracy on independent test data (FIG. 15a; Table 6 in Appendix) and correctly identified from single-lane data all 18 cases where sequencing 6 lanes of a library did not yield 50× coverage. A receiver-operating characteristic (ROC) curve showed a wide-range of favourable operating points (FIG. 15a; area under the curve, AUC=0.993). Analysis of variable importance highlights the value of quality metrics from different categories (FIG. 15b), with metrics of each type contributing. Models for normal data were less accurate, possibly because due to fewer training data (FIG. 16; Table 7 in Appendix): a tumour classifier trained with only 4 lanes of sequencing per sample demonstrated a reduced accuracy of 88.9% (FIG. 17).

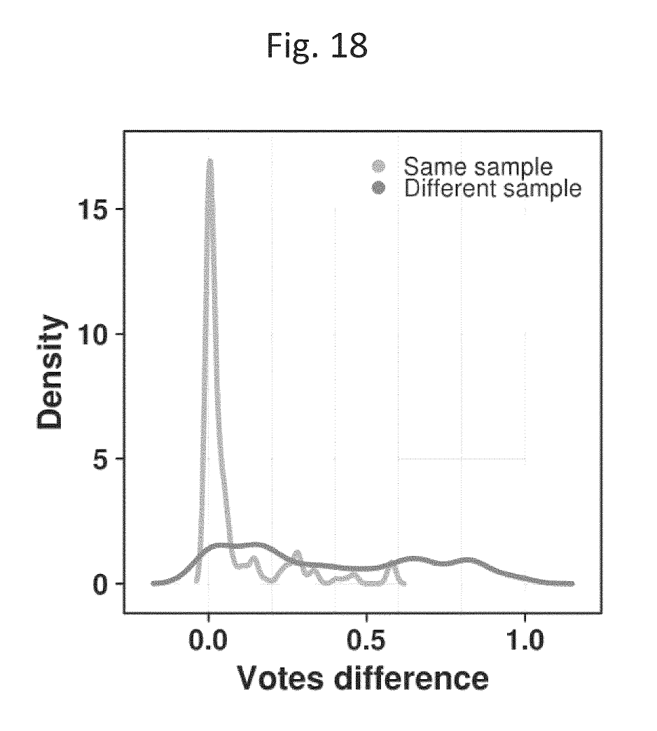

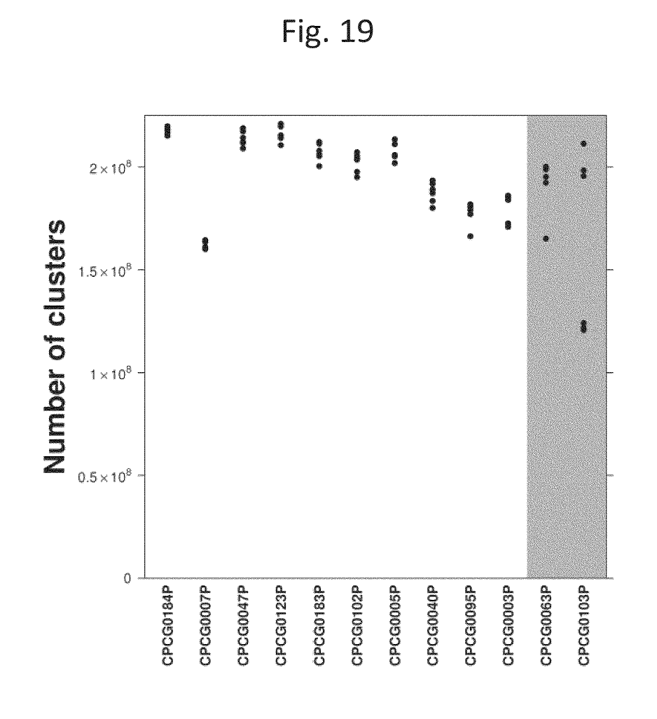

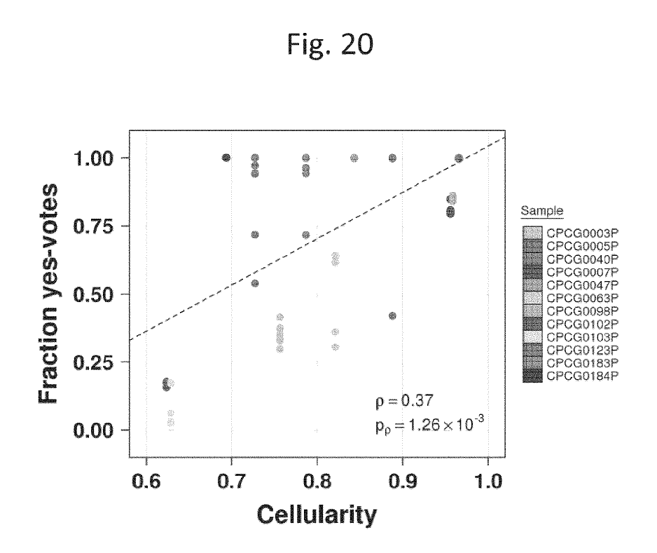

We considered the entire set of predictions for the tumour data by the confidence of our classifier (number of yes votes from the forest), where more homogeneous results (close to 0 or 1) indicate higher confidence (FIG. 15c). While lanes within a sample generally yielded similar predictions (FIG. 18), some showed more variation (e.g. CPCG0063P). This is likely due to inconsistencies in the amount of input DNA: the number of clusters per lane was unusually variable for lanes in this sample (FIG. 19). All FFPE lanes were correctly classified. Intriguingly, the fraction of votes was weakly correlated to sample cellularity (ρ=0.36; p<0.01; FIG. 20), suggesting low cellularity samples tend to yield less usable sequence per lane. This is consistent with the tumour-normal differences (FIG. 7c; FIG. 8), and shows the complexity of relationships that can be revealed by quality-control methods.

Can Protocol Variations Be Detected From Quality Data?

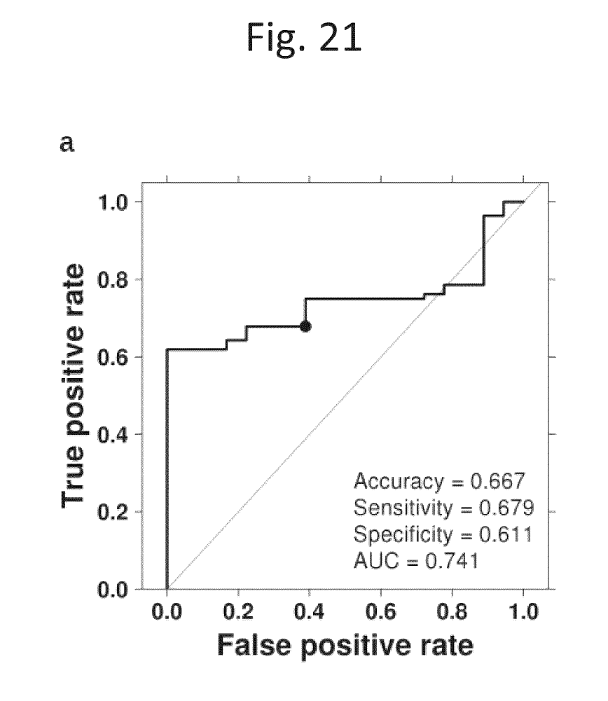

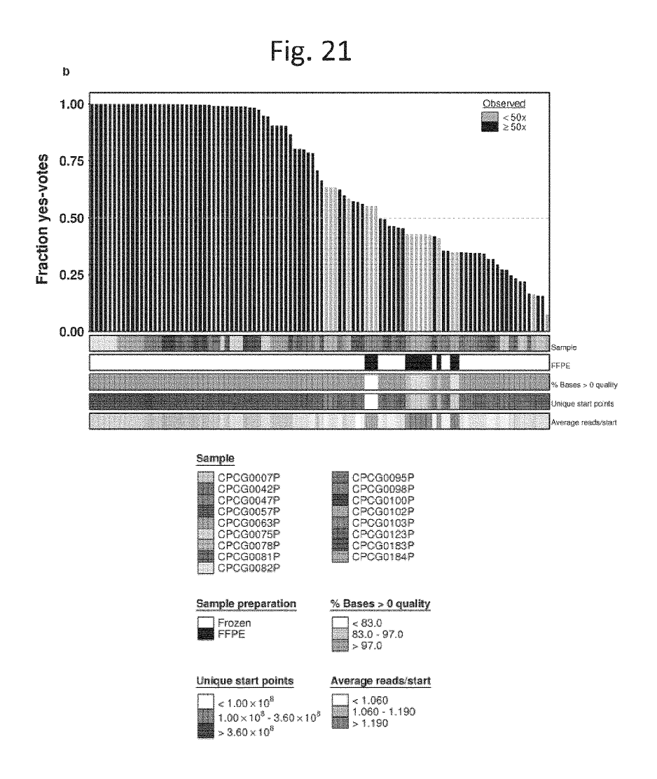

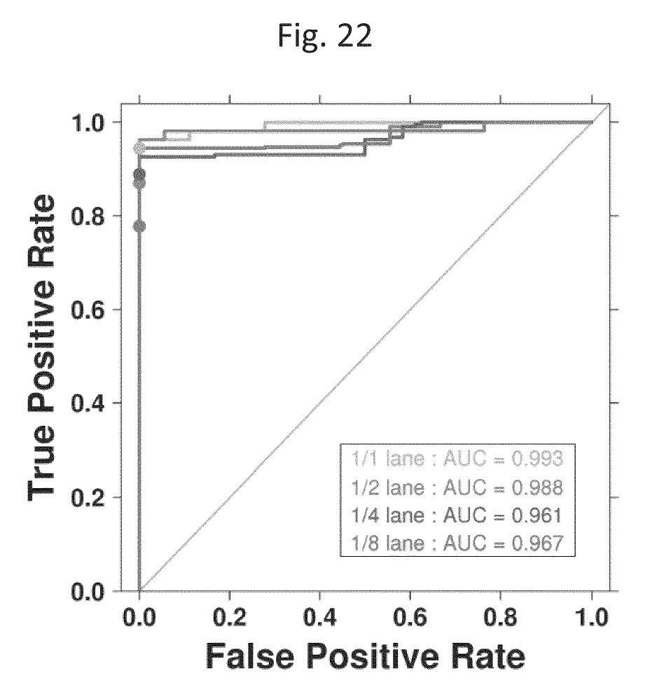

Next we sought to determine if quality metrics diverge over time in a large sequencing centre, as might be caused by technician-specific bias, protocol refinements or reagent-batch differences. We split our 27 tumour genomes into two groups based on sequencing-date, training a model with the 10 oldest samples (See Table 4 in Appendix) and validating on the 17 newest (See Table 5 in Appendix). This age-dichotomized model was significantly less accurate (FIG. 21; AUC_{age}_{_}_{dichotomized}=0.741; AUC_{randomized}=0.993; p=3.0×10⁻⁵), highlighting the importance of routine quality-metric collection for regular re-learning of process-control models. Reagent batches and other experimental details could be used as model covariates to identify experimental features associated with data quality changes.

Can Accurate Predictions Be Made From Even Smaller Amounts of Sequencing?

Our initial analysis shows high accuracy using ˜16-25% of the total data for modelling (i.e. a complete lane). However, for routine production use it would be highly advantageous to use even smaller subsets so multiple samples could be evaluated simultaneously using barcoded libraries. Combined with same-day sequencing instruments, this would allow for routine quality-assessment prior to full-scale sequencing, reducing cost and increasing quality. To identify the minimum amount of data required for accurate predictions, we trained SeqControl with a fraction of a lane of sequencing then validated its prediction ability as before (Tables 8-10 in Appendix). Performance was only modestly decreased when the amount of training sequence changed from a full-lane (AUC=0.993) to an eighth of a lane (AUC=0.967; FIG. 4; Table 11). This suggests that that pooled barcoded libraries can be utilized for routine quality prediction.

TABLE 11
Performance of random forest classifiers trained with 1/k-lane dilution data.
AUC value represents the area under a receiver operating curve.

| k | Accuracy | Sensitivity | Specificity | AUC |
|---|---|---|---|---|
| 2 (half-lanes) | 0.903 | 0.870 | 1.00 | 0.988 |
| 4 (quarter-lanes) | 0.917 | 0.889 | 1.00 | 0.961 |
| 8 (eighth-lanes) | 0.833 | 0.789 | 1.00 | 0.967 |

Samples

The sample DNA analyzed in this study came from the Canadian Prostate Cancer Genome Network (CPC-GENE) project (http://icgc.org/icgc/cgp/70/392/70542). Data was collected from twenty-six prostate cancer patients and consisted of tumour tissue DNA and paired DNA extracted from normal blood samples (referred to as normal DNA) for each patient. In addition, one unmatched tumour sample was also included for which normal data was unavailable. Twenty-five of the tumour samples were prepared via flash-freezing and the remaining two were prepared by formalin-fixation and paraffin-embedding (FFPE). Fresh-frozen post-RP (radical prostatectomy) specimens were from the University Health Network BioBank. FFPE tissue blocks were obtained from the Department of Pathology, University Health Network. Blood samples were collected at the time of informed consent, which followed local Research Ethics Board (REB) and ICGC guidelines (UHN REB study protocols UHN 06-0822-CE and UHN 11-0024-CE).

To estimate the cellularity and purity of tumour samples, we hybridized an aliquot of DNA from each sample to an OncoScan Affymetrix microarray. To calculate tumour purity from this data, we used the qpure algorithm³², which requires Log R Ratio (LRR) and B allele frequency (BAF) for each probe. These were computed using the two intensity values for each probe (i.e. one for each allele interrogated at each position) using the equations: LRR=log₂(X+Y) and BAF=Y/(X+Y), where Y and X are intensity values corresponding to the minor and major alleles, respectively. We used the tumorpurity.mixture.gam.adjust output of qpure as our estimate of cellularity.

Sequencing

Pico-green quantified gDNA (50 ng) was sheared to 300 bp using a Covaris S2 Ultra-sonicator (Covaris Inc., Woburn, Mass., USA) followed by 3× volume AMPure XP SPRI bead clean-up (Beckman Coulter Genomics, Danvers, Mass., USA Cat#A63881). The resulting bead-DNA mixture was transferred to a 96-well PCR plate and libraries were constructed using enzymatic reagents from KAPA Library Preparation Kits (KAPA Biosciences, Woburn, Mass. USA Cat#KK8201) according to previously reported protocols for end repair, A-tailing and adapter ligation³³. Adapter-ligated libraries were enriched by adding 3 μL of 25 μM !lumina F & R PE enrichment primers (Integrated DNA Technologies, Coralville, Iowa, USA), 75 μL of 2× KAPA HiFi HotStart ReadyMix (KAPA Biosciences, Woburn, Mass., USA Cat#KK2602) and 33 μL of nuclease-free water (Life Technologies, Carlsbad, Calif., USA Cat#AM993) to 36 μL of eluted DNA and amplified across 3 individual PCR reaction tubes. Verti 96-well Thermal Cyclers (Life Technologies, Carlsbad, Calif., USA) were used to incubate libraries (45s at 98° C.) and cycled 10 times for 15s at 98° C., 30s at 65° C. and 30s at 72° C. Following a 0.6× SPRI bead clean-up, post-PCR enriched libraries were eluted in 40 μL of elution buffer (Qiagen, Hilden Germany, Cat#19086) and validated using an Agilent Bioanalyzer (using the High Sensitivity DNA Kit; Agilent Technologies, Santa Clara, Calif., USA Cat#5067-4626).

Libraries were quantified on an Illumina Eco Real-Time PCR machine (Illumina Inc., San Diego, Calif., USA) using KAPA Illumina Library Quantification Kits (KAPA Biosciences, Woburn, Mass., USA Cat#KK4835). Paired-end sequencing of 2×101 cycles was carried out for all libraries on the Illumina HiSeq 2000 platform (Illumina Inc., San Diego, Calif., USA). For normal sample CPCG0004R, six lanes were sequenced. For all other normal samples, four lanes were sequenced. All tumour samples had six lanes of sequencing (FIG. 2; Table 1).

Alignment and Pre-Processing

All sequencing was performed on Illumina HiSeq 2000 machines, with raw base-call and intensity files transferred to network storage during sequencing. FASTQ files were generated using IIlumina's CASAVA (v1.8.2), then aligned to the UCSC hg19 human reference (with no repeat-masking) using the Novoalign short-read aligner (v2.07.14; http://www.novocraft.com/). Table 3 lists the parameter values used for this and other processing steps. Novoalign produced output in SAM format³⁴ with properly-configured read groups generated by the Picard tool suite (v1.41; http://picard.sourceforqe.net/). These files were converted to BAM format, sorted by coordinate and indexed using Picard (v1.41). All lane-level BAM files were then filtered individually using SAMtools³⁴ (v0.1.18) to remove unmapped reads and reads mapping to multiple genomic locations (Table 3).

To create data sets consisting of varying numbers of lanes, the lane-level BAM files were grouped. For the first ten patients, we generated all possible lane groupings: every individual lane, every pair of lanes, every set of three lanes, etc. (FIG. 3; Table 1). For each grouping, the lane-level BAMs were merged using Picard's MergeSamFiles function (v1.41) to generate a BAM file containing reads from all lanes in the group. These merged group-level BAM files were then collapsed to remove duplicate reads using Picard's MarkDuplicates function (Table 3), which defines duplicates as reads with the same coordinates for both paired ends. For the other seventeen patients only single-lane and all-lane groupings were generated (Table 1).

Quality Metrics

In order to assess the quality of the sequencing data, a list of 15 quality metrics was generated (Table 2). These metrics were evaluated for each grouping using a custom Perl wrapper script. Uncollapsed coverage, collapsed coverage, number of clusters, number of unique start points and average reads per start point were calculated directly from the group-level BAM files (see above).

To determine how many bases were covered at or above various depths, the BEDTools software suite³⁵ (v2.11.2) was used (Table 3). To account for unknown bases in the reference genome, the coverage files were masked—that is, adjusted so that each unknown (‘N’) base in the UCSC hg19 FASTA files would have a coverage value of NA instead of zero. The resulting files were used to find masked coverage across all known bases in the genome.

To generate information about variant calling confidence, a slightly edited version of SAMtools (v0.1.18) was used. The original source code was changed so that the output file generated by the bcftools view command contained only the CHROM, POS and QUAL fields instead of a full VCF file to reduce computing time and disk space (approximately 4-fold savings). The SAMtools mpileup function (v0.1.18) was run on each group-level BAM and the results were piped to the edited bcftools view function in order to obtain a genotype quality score for each base in the genome (Table 3). This score represents the Phred-scaled probability that the base called is incorrect, so that higher quality scores indicate higher confidence calls

(http://www.1000genomes.org/wiki/Analysis/Variant%20Call%20Format/vcf-variant-call-format-version-40).

Metric values for all group-level BAMs can be found in Table 4 (oldest 10 samples) and Table 5 (newest 17 samples).

Linear Modelling

Linear modelling was performed in R (v3.0.1) and used the lm function of the base stats package. The models were evaluated on testing data using the predict function of the stats package.

Note that for the dotmap in FIG. 2a, training and testing data was standardized within each metric type. For each metric, the mean and standard deviation of all measured values was calculated. Individual values then had the mean subtracted and were divided by the standard deviation to yield the data used for training. This step was performed in order to generate models with more comparable coefficient values for presentation. In contrast, the linear model used in FIG. 2b used un-processed data for training and testing input.

Univariate & Principal Components Analysis

Univariate analysis and PCA were performed using all lanes of the 15 tumour training samples. For each metric, a VVilcoxon rank-sum test between lanes from the two outcome classes (<50× vs. ≧50× collapsed coverage) was run using the wilcox.test function in the R base stats package (v3.0.3), and the p-values were extracted. PCA was performed on metric data from all lanes using the prcomp function of the base stats package (v3.0.3).

Random Forest Classification

Random forests²⁸ were trained using the randomForest package (v4.6-7) in R (v3.0.1). Input variables consisted of all metric values for a single-lane group and outcomes consisted of binary (0 or 1) values representing whether the corresponding all-lane grouping (4-lane for normal, 6-lane for tumour) for this sample achieved the specified target coverage depth (30× for normal, 50× for tumour). The forests were grown to 100,000 trees, and all other parameters used default settings. The trained forests were used to classify testing data with the predict function. See Table 12 for a full list of parameters used. Results from random forest training and testing, including the metric data used for input, can be found in Table 6 (in the Appendix) (tumour) and Table 7 (in Appendix) (normal).

TABLE 12
Command line options specified for training random forests with the randomForest
package. Any parameters not listed below used default values.

| Processing Step | Software Used | Function Used | Parameters Specified |
|---|---|---|---|
| Train random forest | randomForest | randomForest | formula=outcome ~. |
| | package (v4.6-7) | | ntree=100000: train 100,000 |
| | R(v3.0.1) | | trees for the forest |
| | | | importance=TRUE: assess |
| | | | variable importance |
| | | | locallmp=TRUE: assess |
| | | | variable importance case- |
| | | | wise |
| | | | proximity=TRUE: get |
| | | | information about row |
| | | | proximities |
| | | | keep.inbag=TRUE: keep a |
| | | | list of samples used in each |
| | | | tree |
| Test random forest | randomForest | predict | object=trained_forest: object |
| | package (v4.6-7) | | of class randomForest |
| | R(v3.0.1) | | (returned by the |
| | | | randomForest function) |
| | | | newdata=test_data: metric |
| | | | data for the testing sample |
| | | | lanes |
| | | | type=“vote”: output is a matrix |
| | | | containing the fraction of |
| | | | votes for each class |
| | | | AND |
| | | | type=“response”: output is a |
| | | | matrix of 0/1 values for each lane |

For assessing variable importance, the cforest function of the party package (v1.0-13, R v3.0.3) was used. In contrast to the randomForest package, this method accounts for correlations between variables in its forest training and variable importance measures, and therefore gives an unbiased estimate of importance in our classifiers³⁶. For consistency with the randomForest package, an mtry value of 3 was used in model training. See Table 13 fora full list of parameters used.

TABLE 13
Command line options specified for training random forests (cforests) with the party
package. Any parameters not listed below used default values.

| Processing Step | Software Used | Function Used | Parameters Specified |
|---|---|---|---|
| Set random forest arguments | Party package | cforest_control | ntree=100000: train 100,000 trees for the forest |
| | (v1.0-13) | | mtry=3: number of variables selected for splitting at each |
| | R (v3.0.3) | | node |
| Train random forest | Party package | cforest | formula=outcome ~. |
| | (v1.0-13) | | |
| | R (v3.0.3) | | |
| Test random forest | Party package | predict | object=trained_forest: object of class RandomForest |
| | (v1.0-13) | | (returned by the cforest function) |
| | R (v3.0.3) | | newdata=test_data: metric data for the testing samplelanes |
| | | | type=“prob”: outcome is a matrix of probabilities for each class |
| | | | AND |
| | | | type=“response”: output is a matrix of 0/1 values for each |
| | | | lane |

ROC Comparison

The pROC R package³⁷ (v1.5.4) was used to calculate a p-value representing the significance of the difference between two ROC curves. The comparison was performed with the roc.test function using Venkatraman's test for unpaired ROC curves with 100,000 bootstraps. All other parameters used default settings.

Dilution Analysis

To split each individual BAM file we used Java (v1.6.0_25), Picard (v1.92), and the Rsamtools package (v1.14.3) in the R statistical environment (v3.0.3). Table 14 shows all the parameters used in this step.

TABLE 14
Command line options specified for the BAM splitting steps.
Any parameters not listed below used default values.

| Processing Step | Software | Function Used | Parameters Specified |
|---|---|---|---|
| Build BAM Index | Java | BuildBamIndex | I=in_bam: BAM input directory |
| | (v1.6.0_25) | | O=out_bam: BAM index output directory |
| | Picard (v1.92) | | |
| Generate Unique | R (v3.0.3) | ScanBamParam | what=qname: query name; i.e., identifier, associated with the |
| Reads | Rsamtools | | read |
| | (v1.14.3) | | flag=ScanBamFlag( |
| | | | isFirstMateRead=TRUE: whether the first mate read |
| | | | should be returned |
| | | | isSecondmateRead=FALSE: whether the second mate read |
| | | | should be returne |
| | | | isNotPrimaryRead=FALSE: whether alignments that are |
| | | | primary) |
| | | scanBAM | param=ScanBamParam( |
| | | | what=”qname” |
| | | | flag=ScanBamFlag( |
| | | | isFirstMateRead=TRUE, |
| | | | isSecondmateRead=FALSE: |
| | | | isNotPrimaryRead=False)) |
| Creat BAM file | Java | FilterSamReads | I=input_bam: BAM file to be filtered. |
| | (v1.6.0_25) | | O=output_bam: BAM file to write read excluded results to. |
| | Picard (v1.92) | | READ_LIST_FILE=out_file: Read List File containing reads |
| | | | that will be included or excluded from the ouput BAM file. |
| | | | FILTER=includeReadList: output BAM will contain reads that |
| | | | are supplied in the READ_LIST_FILE file. |
| | | | SORT_ORDER=coordinate: SortOrder of the output BAM file |
| | | | WRITE_READS_FILES=false: do not create .read files |

For each BAM file we first created a BAM index file (.bai) using Picard's BuildBamIndex function. We then created k files such that each file contained 1/k unique read IDs, sampled at random, without replacement, from the original full-lane BAM. From each of the k files we then created a new BAM file representing the subset of reads using Picard's FilterSamReads function (Table 14). The resulting set of k files represents all the reads from the full-lane BAM partitioned into k subsets. This was repeated for k=2, 4, and 8 to generate data for half-lanes, quarter-lanes, and eighth-lanes. Quality metric values for these partial-lane BAMs were generated using the same methods described for full-lane data.

Random forest analysis was repeated using dilution data for training and testing. Results of this analysis are summarized in Table 11.

Visualization

All plots were created using custom R scripts executed in the R statistical environment (v3.0.1, v3.0.3). Plots were drawn using the lattice (v0.20-15, v0.20-28) and latticeExtra (v0.6-24) packages.

Data Access

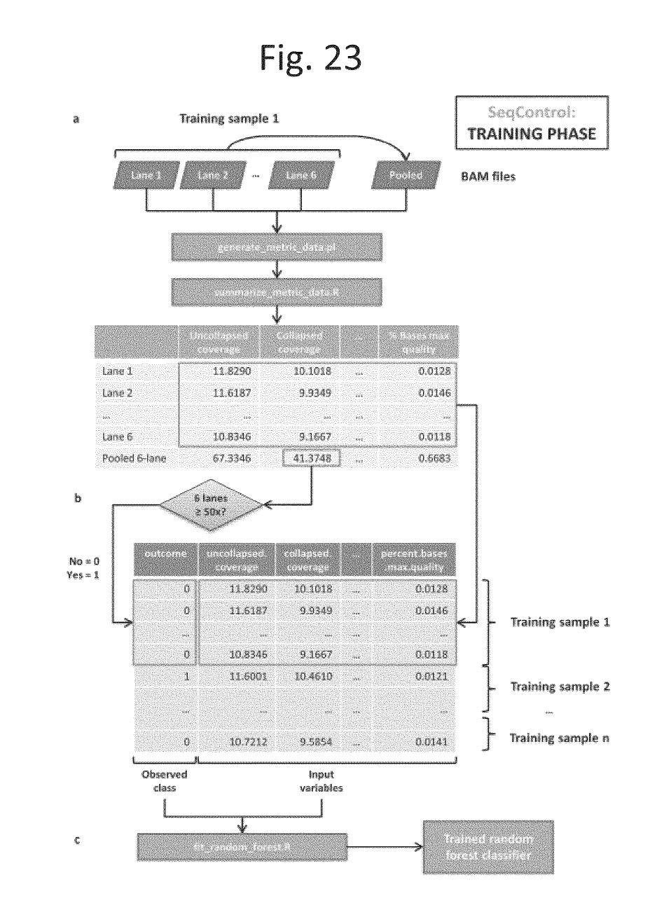

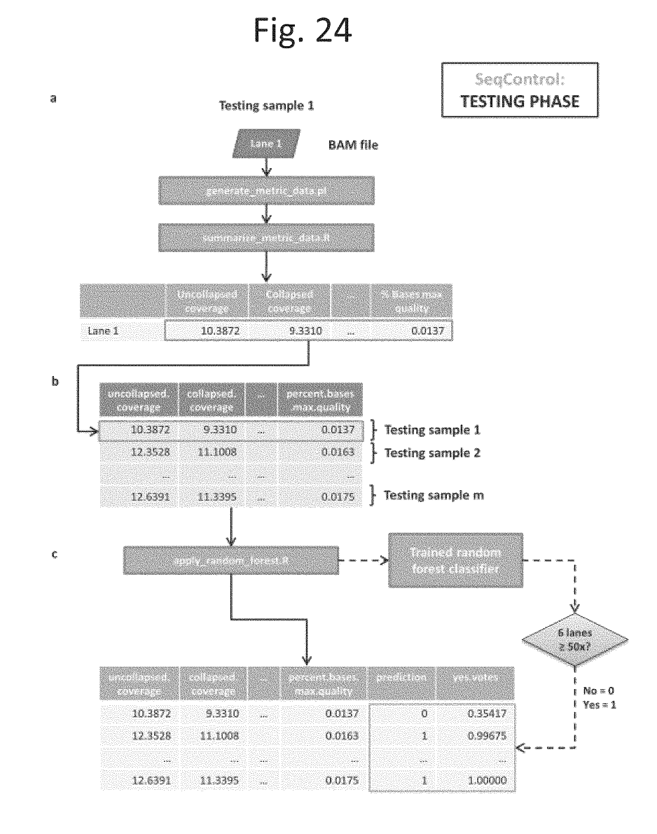

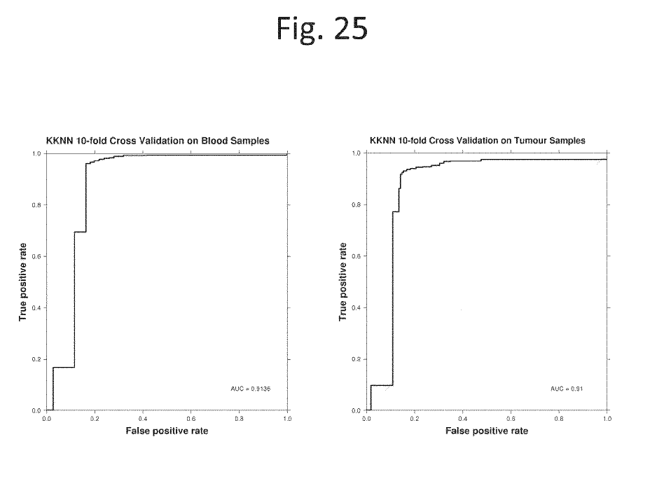

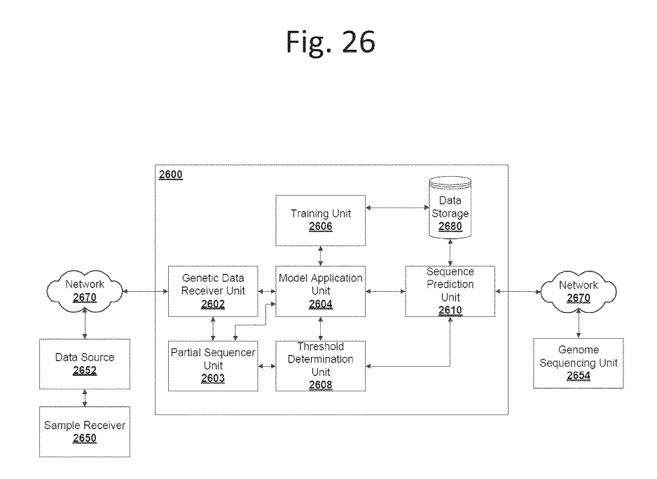

Metric values for all groupings are located in Table 4 and Table 5. For the original ten samples (Table 4), all groupings of every size were generated and processed for both normal and tumour tissue. For the additional seventeen samples (Table 5), only single-lane and all-lane data (4-lane for normal, 6-lane for tumour) was generated and processed. Partial-lane data for the 17 tumours can be found in Tables 8, 9, and 10 (in Appendix) (half-lane, quarter-lane, and eighth-lane, respectively). All raw sequencing data will be deposited in the European Genotype-Phenotype Archive (EGA study accession number: EGAS00001000573) and array data will be deposited in GEO. All code for generating SeqControl metric value and training/testing random forests can be downloaded as SeqControl package v0.0.1. FIGS. 23 & 24 depict the general workflow for training and testing.

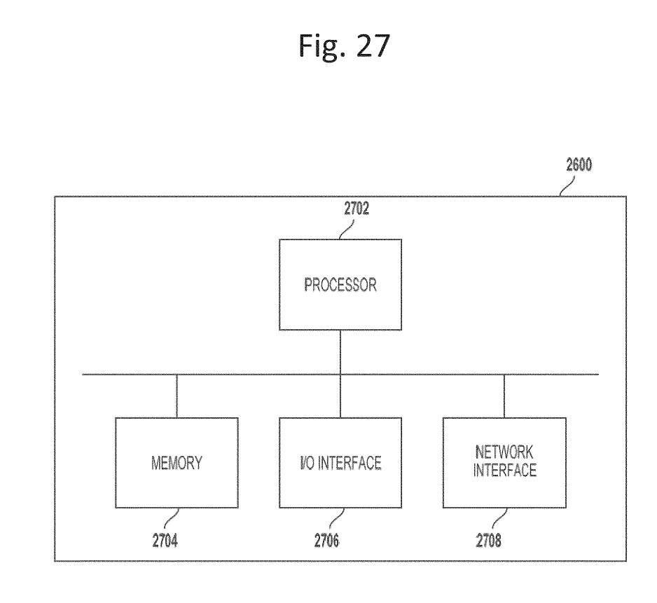

A weighted k-nearest neighbours model was tuned, and revealed that using k=2 and a distance of 2 with a triangular kernel yielded the best model. The space was tuned over of k from 1 to 100 and distance from 1 to 100. Ten fold cross validation was performed on the tuned model to create the ROC curves provided. This was an analysis of 508 lanes of normal and 843 lanes of tumour using the full set of expanded metrics (FIG. 25).

One of the major challenges in predicting sequencing quality is variability in library complexity. While the definition of library complexity may vary slightly between applications, it is generally defined as the number of unique DNA fragments in the library (i.e. the number of fragments collected from the original sheared input DNA). In sequencing context, complexity is sometimes described as the unique reads contained in the total reads from an experiment.

Multiple factors can influence library complexity. The amount of starting DNA is critical in the fragmentation and size selection steps. Since fragmentation is random, an insufficient amount of input DNA leads to a decreased chance of obtaining a fragment of the desired length that spans any given region. Low input volume leads to a library containing fragments from only a small portion of the overall target region, so sufficient starting DNA is critical to generating a high-complexity library. This can be a serious limitation for applications like cancer where the available sample material is often limited. The PCR step is another major source of variability. Bias towards some fragments being over-amplified (for example as caused by GC-content, fragment length, etc.) leads to the under-amplification of others. This results in an overall imbalance and reduces the number of well-represented regions. Consequently the number of PCR cycles performed is critical to creating complex libraries, and this design decision needs to be optimized according to the specific application. The significant bias introduced by the PCR step has led to an increasing focus on amplification-free protocols in recent years. These show promising results but are still in their infancy for human whole-genome sequencing applications.

The inherent variability in library complexity introduced by these factors makes quality prediction challenging, since all libraries have different characteristics and behave differently as additional sequencing is performed. Some researchers create multiple libraries for a single sample that can either use the same or different target fragment size in order to account for the random biases. Other issues cannot be avoided, and this highlights the need for a method to characterize and predict quality on a per-library basis.

What is the Intra- and Inter-Sample Variance in Quality?

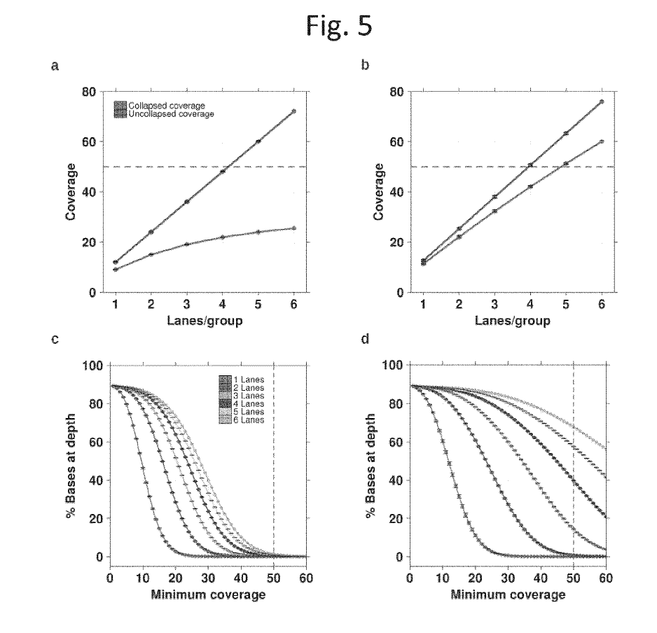

To examine the variability in the metric data, we initially focused on 10 tumours and 9 normal references (i.e. the first ten patients analyzed; Table 4). In all cases, we observed marked differences in the quality metrics both within a sample (between lanes) and between samples (FIG. 4). Inter-sample variability was not eliminated by adding sequencing. For instance, the tumour library for sample CPCG0001 has low complexity: additional lanes minimally altered overall collapsed coverage due to the large proportion of duplicate reads (FIG. 5a). By contrast, the tumour library for CPCG0020 showed a steady increase in collapsed coverage with additional lanes (FIG. 5b), indicating higher library complexity. The low-complexity CPCG0001 library also showed a lower percentage of individual bases reaching any given coverage depth than CPCG0020 (FIGS. 5c & 5d). These data reflect the heterogeneity seen in large-scale sequencing projects, and highlight the significant variability present in real data.

Are Different Quality Metrics Related?

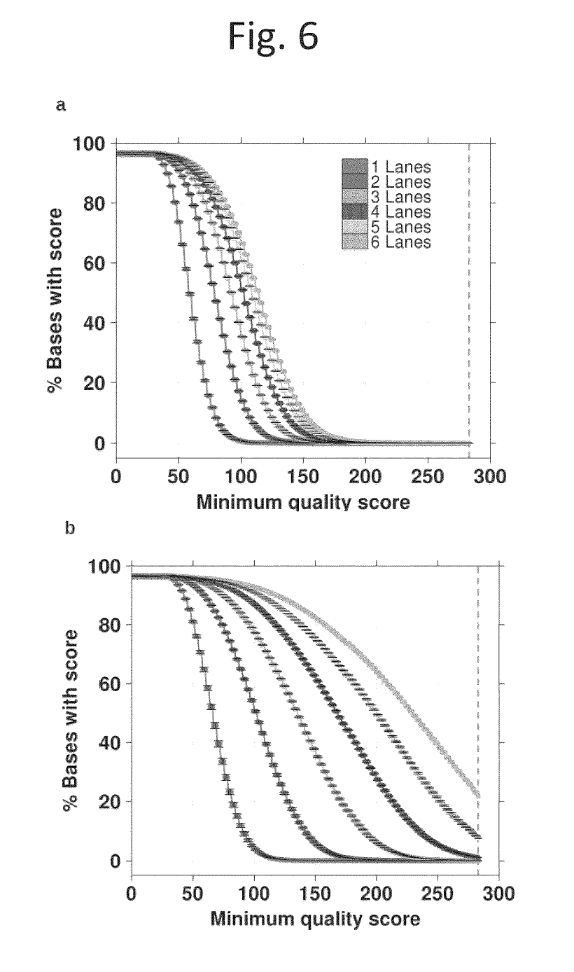

The information content of additional sequencing is non-linearly dependent on several factors, including the number of lanes sequenced, the number of unique molecules in a library and lane-to-lane variability in sequencing, and eventually saturates in both coverage (FIG. 5) and quality (FIG. 6). We therefore compared every pair of quality metrics to determine if they contain unique information, or are extremely highly-correlated and reflect a single underlying trend. We calculated Spearman's rank correlation coefficient (ρ) pairwise using values from all groupings, considering normal (FIG. 7a) and tumour samples (FIG. 7b) separately. In both tissues, all pairs were strongly, but imperfectly correlated (0.70<ρ<1; p<10⁻⁷⁰). Metrics that do not reflect data redundancy (e.g. uncollapsed coverage) or that do so weakly (e.g. percentage of bases with greater than 0× coverage) showed moderate correlations with those that do (e.g. collapsed coverage, percentage of bases with greater than 50× coverage). On the other hand, as expected a subset of metrics showed strong correlations: high-coverage and high-quality bases are correlated as additional reads yield higher-confidence genotype calls.

Interestingly, normal and tumour samples show divergent correlation profiles. The difference between the two profiles (FIG. 7c) shows that most correlations are weaker in tumour (i.e. almost all differences are positive), which may reflect fundamental differences such as cellular heterogeneity within tumours. However, it may also be a result of the larger amount of tumour sequence: metrics that measure the proportion of high-coverage bases (e.g. 30×, 50×) are noisier in normal data due to lower average coverage.

How Well Do Existing Tools Predict Sequencing Quality?

A recently developed tool, preseq⁷, is the state-of-the-art for predicting library complexity. Given a small sub-experiment, it uses a non-parametric empirical Bayes method to predict complexity—measured as the number of unique reads present in a total number of reads sequenced. We applied preseq to all 162 lane-level BAMs from our twenty-seven tumour samples (Table 15 in Appendix). Of these, 52% (85/162) failed because preseq was unable to calculate bootstrap-based 95% confidence intervals. The initial report of preseq suggested this as a typical challenge for low-quality data. Our samples showed similar characteristics to other samples at our centre, although with smaller insert-sizes due to the low-input library preparation protocol used (50 ng input DNA). Interestingly, even on the 77/162 successful lanes, preseq results differed significantly from our observed results (FIG. 12a). We traced this to a discrepancy in the definition of a unique read. Preseq identifies duplicates using only the start coordinates of individual reads, whereas the Picard method we used incorporates the start coordinates of both reads in a pair. This causes a larger proportion of reads to be identified as duplicates⁸ in preseq, leading to underestimation of the unique reads. The preseq predictions were thus better compared with our metric for unique start points (FIG. 12b). Even with this revised comparison, individual lanes with particularly high or low quality led to preseq predictions with poor correlation to our observed 6-lane data (FIG. 12c). In contrast, for uniform sequencing runs preseq performed very well (FIG. 12d-f). Of the 77 lanes that yielded predictions, 47% (36/77) gave preseq-estimated 95% confidence intervals that contained the true outcome value. Thus, the state-of-the-art tool could not be run for 52% of our samples and showed poor predictive accuracy, especially for challenging samples—exactly the real-world cases where a quality-control model is most needed.

The definition of library complexity differs according to how duplicate reads are defined (i.e. single-end or paired-end), and this represents the main difference we see between preseq and our results. This discrepancy could easily be accounted for with considered analysis of the preseq results.

However, a more serious limitation is that it is not trivial to deduce how many sequencing lanes are required to yield a desired total number of reads. Our metric data shows that the total number of reads in a lane can vary widely, even within a sample. For experimental design, there is value in determining the actual amount of sequencing that should be performed.

Correlations

Metric values for all groups were read into the R statistical environment (v3.0.1). All Spearman correlations were calculated using the cor function of the base stats package (v3.0.1). Clustering in heatmaps was performed using the diana function of the cluster package (v1.14.4) which implements the divisive analysis clustering technique.

Preseq Testing

The preseq tool7 (v0.0.1) was tested by downloading and installing the open-source software. Individual lane-wise BAM files that were filtered according to our pipeline (Table 3) were used as input to the Ic_extrap function to produce predicted complexity curve values for each lane. The predicted number of unique reads in the corresponding 6-lane data was deduced from the Ic_extrap output using the observed total reads. These predictions were then compared to the observed unique read counts. Complexity curves for the lane-level and 6-lane BAMs were also generated for plotting purposes. All non-default input settings used are listed in Table 16.

TABLE 16
Command line options specified for pre5eq²³ testing.
Any parameters not listed below used default values.

| Processing Step | Function Used | Parameters Specified |
|---|---|---|
| Generating | c_curve | B: input is in BAM format |
| complexity | | v: verbose |
| curves for | | |
| existing lane- | | |
| level and 6-lane | | |
| merged BAMs | | |
| Generating | lc_extrap | B: input is in BAM format |
| predicted | | v: verbose |
| complexity using | | e 3000000000: generate yield |
| lane-level input | | estimates for up to 3 billion total |
| BAMs | | reads |

Discussion

Data quality remains a critical issue in sequencing studies. Failure to meet pre-planned coverage thresholds is common: in the CPC-GENE ICGC project, 24% of normal samples and 25% of tumours did not initially reach target depth and required “top-up” sequencing. This imprecision may be acceptable in research settings, but not when sequencing is a component of clinical and industrial processes. For such applications, systematic strategies for evaluating and predicting data quality are needed. We demonstrate the viability of using statistical process control techniques for next-generation sequencing to refine and enhance existing sequencing pipelines. However, systematic retrospective evaluations are computationally challenging: over 260 TB and 88,000 CPU hours was invested into this study.

Therefore a first key step for most centres will be modifying existing Laboratory Information Management Systems (LIMS) and pipelines to routinely collect and store quality metrics. Over time, prospective data-collection will create large databases without the costs of retrospective analyses. Periodic model re-training will facilitate detection of changes in error profiles (FIG. 21). Incorporation of experimental meta-data (i.e. protocol, reagent batch, technician, tumour cellularity, etc.) will be particularly valuable, as this may allow for real-time quality monitoring. The lack of consistent LIMS software and data-exchange interfaces greatly complicates the exchange of data like this across centres. It would therefore be of significant benefit to the community if a global database of sequencing quality metrics was developed: instrument vendors are ideally placed to facilitate this.

While our approach is general, some models may need retraining for new platforms and experimental protocols. For example, the SeqControl methodology could be applied to targeted sequencing data (e.g. exome or ChIP-Seq), but each library construction protocol may require its own model. Similarly, each application may require specific tuning: 50× coverage is common today but some studies sequence to 200×²⁹ or deeper^{30,31}. New technologies will have different error and performance characteristics, necessitating development of new control models during initial work-up. Input sample type (e.g. different tissue or tumour types) may also influence sequencing results: the correlation of classifier votes with tumour cellularity hints at this. Fortunately, Random Forests can be trained in a few CPU minutes. SeqControl is open-source and can be easily extended with novel statistical and feature-selection approaches that incorporate these additional covariates. This flexibility underlies the wide range of research and commercial applications that can benefit from the use of predictive quality control.

The challenge of accurately predicting outputs of a complex system given varied and incomplete information about the inputs is common in industrial engineering. For NGS data, as in other industries, the solution will likely involve the application and extension of techniques from the fields of control theory and statistical process control. While the prediction of next-generation sequencing data quality is an emerging field, our results suggest there is untapped value in retaining and analyzing quality metric data. SeqControl represents rigorous control and optimization of next-generation sequencing.

It will be appreciated by those skilled in the art that other variations of the embodiments described herein may also be practiced without departing from the scope of the invention. Other modifications are therefore possible.

Although the disclosure has been described and illustrated in exemplary forms with a certain degree of particularity, it is noted that the description and illustrations have been made by way of example only. Numerous changes in the details of construction and combination and arrangement of parts and steps may be made. Accordingly, such changes are intended to be included in the invention, the scope of which is defined by the claims.

Except to the extent explicitly stated or inherent within the processes described, including any optional steps or components thereof, no required order, sequence, or combination is intended or implied. As will be understood by those skilled in the relevant arts, with respect to both processes and any systems, devices, etc., described herein, a wide range of variations is possible, and even advantageous, in various circumstances, without departing from the scope of the invention, which is to be limited only by the claims. All references and publications mentioned in this specification, including in the following reference list, are hereby incorporated by reference.

### REFERENCES

1. 1000 Genomes Project Consortium et al. A map of human genome variation from population-scale sequencing. Nature 467, 1061-1073 (2010).
2. Meyer, M. et al. A high-coverage genome sequence from an archaic Denisovan individual. Science 338,222-226 (2012).
3. Lachance, J. et al. Evolutionary history and adaptation from high-coverage whole-genome sequences of diverse African hunter-gatherers. Cell 150, 457-469 (2012).
4. Prufer, K. et al. The bonobo genome compared with the chimpanzee and human genomes. Nature 486,527-531 (2012).
5. Huang, X. et al. A map of rice genome variation reveals the origin of cultivated rice. Nature 490, 497-501 (2012).
6. Groenen, M. A. M. et al. Analyses of pig genomes provide insight into porcine demography and evolution. Nature 491,393-398 (2012).
7. D'Hont, A. et al. The banana (Musa acuminata) genome and the evolution of monocotyledonous plants. Nature 488,213-217 (2012).
8. Paterson, A. H. et al. The Sorghum bicolor genome and the diversification of grasses. Nature 457,551-556 (2009).
9. Fan, H. C. et al. Non-invasive prenatal measurement of the fetal genome. Nature 487,320-324 (2012).
10. Hopf, T. A. et al. Three-dimensional structures of membrane proteins from genomic sequencing. Cell 149,1607-1621 (2012).
11. Cancer Genome Atlas Network. Comprehensive molecular characterization of human colon and rectal cancer. Nature 487,330-337 (2012).
12. Govindan, R. et al. Genomic landscape of non-small cell lung cancer in smokers and never-smokers. Cell 150,1121-1134 (2012).
13. Lupski, J. R. et al. Whole-genome sequencing in a patient with Charcot-Marie-Tooth neuropathy. N. Engl. J. Med. 362,1181-1191 (2010).
14. Bras, J., Guerreiro, R. & Hardy, J. Use of next-generation sequencing and other whole-genome strategies to dissect neurological disease. Nat. Rev. Neurosci. 13,453-464 (2012).
15. Tran, B. et al. Feasibility of real time next generation sequencing of cancer genes linked to drug response: results from a clinical trial. Int. J. Cancer 132,1547-1555 (2013).
16. Wagle, N. et al. High-throughput detection of actionable genomic alterations in clinical tumor samples by targeted, massively parallel sequencing. Cancer Discov 2, 82-93 (2012).
17. Fu, G. K. et al. Molecular indexing enables quantitative targeted RNA sequencing and reveals poor efficiencies in standard library preparations. Proc. Natl. Acad. Sci. U.S.A. 111, 1891-1896 (2014).
18. Clark, M. J. et al. Performance comparison of exome DNA sequencing technologies. Nat Biotech 29,908-914 (2011).
19. Frith, M. C., Wan, R. & Horton, P. Incorporating sequence quality data into alignment improves DNA read mapping. Nucleic Acids Res. 38, e100 (2010).
20. Hower, V., Starfield, R., Roberts, A. & Pachter, L. Quantifying uniformity of mapped reads. Bioinformatics 28,2680-2682 (2012).
21. Ruffalo, M., Koyuturk, M., Ray, S. & LaFramboise, T. Accurate estimation of short read mapping quality for next-generation genome sequencing. Bioinformatics 28, i349-i355 (2012).
22. Tae, H., Ryu, D., Sureshchandra, S. & Choi, J.-H. ESTclean: a cleaning tool for next-gen transcriptome shotgun sequencing. BMC Bioinformatics 13,247 (2012).
23. Daley, T. & Smith, A. D. Predicting the molecular complexity of sequencing libraries. Nat Meth 10, 325-327 (2013).
24. Lewis, F., Maughan, N. J., Smith, V., Hillan, K. & Quirke, P. Unlocking the archive-gene expression in paraffin-embedded tissue. J. Pathol. 195, 66-71 (2001).
25. Lehmann, U. & Kreipe, H. Real-time PCR analysis of DNA and RNA extracted from formalin-fixed and paraffin-embedded biopsies. Methods 25, 409-418 (2001).
26. International Cancer Genome Consortium et al. International network of cancer genome projects. Nature 464, 993-998 (2010).
27. Fieller, E. C., Hartley, H. 0. & Pearson, E. S. Tests for Rank Correlation Coefficients. I. Biometrika 44, 470-481 (1957).
28. Breiman, L. Random Forests. Machine Learning 45, 5-32 (2001).
29. Nik-Zainal, S. et al. The life history of 21 breast cancers. Cell 149, 994-1007 (2012).
30. Chiu, R. W. K. et al. Non-invasive prenatal assessment of trisomy 21 by multiplexed maternal plasma DNA sequencing: large scale validity study. BMJ 342, c7401 (2011).
31. Forshew, T. et al. Noninvasive identification and monitoring of cancer mutations by targeted deep sequencing of plasma DNA. Sci Transl Med 4, 136ra68 (2012).
32. Song, S. et al. qpure: A tool to estimate tumor cellularity from genome-wide single-nucleotide polymorphism profiles. PLoS ONE 7, e45835 (2012).
33. Fisher, S. et al. A scalable, fully automated process for construction of sequence-ready human exome targeted capture libraries. Genome Biol. 12, R1 (2011).
34. Li, H. et al. The Sequence Alignment/Map format and SAMtools. Bioinformatics 25,2078-2079 (2009).
35. Quinlan, A. R. & Hall, I. M. BEDTools: a flexible suite of utilities for comparing genomic features. Bioinformatics 26, 841-842 (2010).
36. Strobl, C., Boulesteix, A.-L., Kneib, T., Augustin, T. & Zeileis, A. Conditional variable importance for random forests. BMC Bioinformatics 9, 307 (2008).
37. Robin, X. et al. pROC: an open-source package for R and S+to analyze and compare ROC curves. BMC Bioinformatics 12,77 (2011).
38. Chen, Y. et al. Systematic evaluation of factors influencing ChIP-seq fidelity. Nat Methods 9,609-614 (2012).
39. Adey, A. et al. Rapid, low-input, low-bias construction of shotgun fragment libraries by high-density in vitro transposition. Genome Biology 11, R119 (2010).
40. Kozarewa, I. et al. Amplification-free Illumina sequencing-library preparation facilitates improved mapping and assembly of (G+C)-biased genomes. Nat Meth 6,291-295 (2009).
41. Aird, D. et al. Analyzing and minimizing PCR amplification bias in Illumina sequencing libraries. Genome Biology 12, R18 (2011).
42. Thompson, J. F. & Milos, P. M. The properties and applications of single-molecule DNA sequencing. Genome Biology 12,217 (2011).
43. Shin, S. C. et al. Advantages of Single-Molecule Real-Time Sequencing in High-GC Content Genomes. PLoS ONE 8, e68824 (2013).
44. Daley, T. & Smith, A. D. Predicting the molecular complexity of sequencing libraries. Nat Meth 10, 325-327 (2013).
45. Bainbridge, M. N. et al. Whole exome capture in solution with 3 Gbp of data. Genome Biology 11, R62 (2010).

### APPENDIX

Lengthy table referenced here
US20170228496A1-20170810-T00001
Please refer to the end of the specification for access instructions.

Lengthy table referenced here
US20170228496A1-20170810-T00002
Please refer to the end of the specification for access instructions.

Lengthy table referenced here
US20170228496A1-20170810-T00003
Please refer to the end of the specification for access instructions.

Lengthy table referenced here
US20170228496A1-20170810-T00004
Please refer to the end of the specification for access instructions.

Lengthy table referenced here
US20170228496A1-20170810-T00005
Please refer to the end of the specification for access instructions.

Lengthy table referenced here
US20170228496A1-20170810-T00006
Please refer to the end of the specification for access instructions.

Lengthy table referenced here
US20170228496A1-20170810-T00007
Please refer to the end of the specification for access instructions.

Lengthy table referenced here
US20170228496A1-20170810-T00008
Please refer to the end of the specification for access instructions.

Lengthy table referenced here
US20170228496A1-20170810-T00009
Please refer to the end of the specification for access instructions.

Lengthy table referenced here
US20170228496A1-20170810-T00010
Please refer to the end of the specification for access instructions.

Lengthy table referenced here
US20170228496A1-20170810-T00011
Please refer to the end of the specification for access instructions.

Lengthy table referenced here
US20170228496A1-20170810-T00012
Please refer to the end of the specification for access instructions.

Lengthy table referenced here
US20170228496A1-20170810-T00013
Please refer to the end of the specification for access instructions.

Lengthy table referenced here
US20170228496A1-20170810-T00014
Please refer to the end of the specification for access instructions.

Lengthy table referenced here
US20170228496A1-20170810-T00015
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20170228496A1). An electronic copy of the table will also be available from the USPTO upon request and payment of the fee set forth in 37 CFR 1.19(b)(3).

1. A method of determining the amount of sequencing required to achieve a target sequencing quality of a genetic sample to be sequenced, the method comprising:
receiving the genetic sample;
sequencing a portion of the genetic sample;
generating from the sequencing a sequencing quality metric, said sequencing quality metric belonging to a category of sequencing quality metrics;
determining the amount of sequencing of the genetic sample required to achieve the target sequencing quality by inputting the sequencing quality metric into a model trained with a plurality of reference sequencing quality metrics to predict sequencing quality.

2. The method of claim 1, wherein the category of sequencing quality metrics is selected from the group consisting of overall coverage, coverage distribution, base-wise coverage, base-wise quality, sequencing experimental information, read-level sequence-quality, read-level mapping-quality, and coverage of genomic repeats.

3. The method of claim 2, wherein the sequencing quality metric belonging to the overall coverage category is selected from the group consisting of uncollapsed coverage, collapsed coverage, masked coverage and clusters. For each of these a summary statistic can be generated such as, but not limited to, the mean, median, standard-deviation, first-quartile and third-quartile.

4. The method of claim 2, wherein the sequencing quality metric belonging to the coverage distribution category is selected from the group consisting of unique start points and average reads/starts.

5. The method of claim 2, wherein the sequencing quality metric belonging to the base-wise coverage category is selected from the group consisting of percentage of bases that reach 0× coverage, percentage of bases that reach 1× coverage, percentage of bases that reach 2× coverage, percentage of bases that reach 3× coverage, percentage of bases that reach 4× coverage, percentage of bases that reach 5× coverage, percentage of bases that reach 6× coverage, percentage of bases that reach 7× coverage, percentage of bases that reach 8× coverage, percentage of bases that reach 9× coverage, percentage of bases that reach 10× coverage, percentage of bases that reach 20× coverage, percentage of bases that reach 30× coverage, percentage of bases that reach 40× coverage, percentage of bases that reach 50× coverage percentage of bases that reach 75× coverage, percentage of bases that reach 100× coverage, percentage of bases that reach 150× coverage, percentage of bases that reach 200× coverage, percentage of bases that reach 250× coverage, percentage of bases that reach 500× coverage, and percentage of bases that reach 1000× coverage.

6. The method of claim 2, wherein the sequencing quality metric belonging to the base-wise quality category is selected from the group consisting of percentage of bases receiving a base-wise genotype quality score greater than 0, percentage of bases receiving a base-wise genotype quality score of at least 10, percentage of bases receiving a base-wise genotype quality score of at least 20, percentage of bases receiving a base-wise genotype quality score of at least 30, percentage of bases receiving a base-wise genotype quality score of at least 40, percentage of bases receiving a base-wise genotype quality score of at least 50, percentage of bases receiving a base-wise genotype quality score of at least 60, percentage of bases receiving a base-wise genotype quality score of at least 70, percentage of bases receiving a base-wise genotype quality score of at least 80, percentage of bases receiving a base-wise genotype quality score of at least 90, percentage of bases receiving a base-wise genotype quality score of at least 100, and percentage of bases receiving the maximum base-wise genotype quality score.

7. The method of claim 2, wherein the sequencing quality metric belonging to the sequencing experimental information category is selected from the group consisting of Machine ID, machine-name, cluster density, technician name or technician ID.

8. The method of claim 2, wherein the sequencing quality metric belonging to the read-level sequence-quality category is selected from the group consisting of average per-read quality, maximum per-read quality, median per-read quality, SD of per-read quality, first-quartile of per-read quality and third-quartile of per-read quality.

9. The method of claim 2, wherein the sequencing quality metric belonging to the read-level mapping-quality category is selected from the group consisting of mapping quality of the read, average mapping quality, median mapping quality, standard deviation of mapping quality, first quartile of mapping quality, third quartile of mapping quality.

10. The method of claim 1, wherein a plurality of sequencing quality metrics are generated.

11. The method of claim 10, wherein the plurality of sequencing quality metrics consists of 2, 3, 4, 5, 6 , 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 sequencing quality metrics.

12. The method of claim 10, wherein the plurality of sequencing quality metrics comprises at least 2 sequencing quality metrics selected from 2 different categories of sequencing quality metrics.

13. The method of claim 10, wherein the plurality of sequencing quality metrics comprises at least 3 sequencing quality metrics selected from 3 different categories of sequencing quality metrics.

14. The method of claim 10, wherein the plurality of sequencing quality metrics comprises at least 4 sequencing quality metrics selected from 4 different categories of sequencing quality metrics.

15. The method of claim 1, wherein the model is trained by inputting the plurality of reference sequencing quality metrics into the model.

16. The method of claim 1, wherein the model comprises a random forest classifier, a neural network, K-nearest neighbours, support vector machines, linear regression, linear discriminant analysis, or decision trees.

17. The method of claim 1, wherein the portion of the genetic sample is greater than 0% and less than 100% of the genetic sample.

18. The method of claim 17, wherein the portion of the genetic sample is less than 50%, less than 40%, less than 30%, less than 20%, less than 10%, less than 5%, or less than 3% of the genetic sample.

19. The method of claim 17, wherein the portion of the genetic sample is at least 1% of the genetic sample.

20. The method of claim 17, wherein the portion of the genetic sample is at least 2% of the genetic sample.

21. The method of claim 17, wherein the portion of the genetic sample is between 2% and 50% of the genetic sample.

22. The method of claim 1, wherein the genetic sample is a genome.

23. The method of claim 1, wherein the genetic sample originates from a tumour genome.

24. The method of claim 1, wherein the genetic sample originates from a non-tumour genome.

25. The method of claim 1, wherein the genetic sample is a targeted sequence of a portion of a genome.

26. The method of claim 25, wherein the targeted sequence of a portion of the genome is an exome.

27. The method of claim 25, wherein the targeted sequence of a portion of the genome is a targeted panel.

28. The method of claim 1, wherein the target sequencing quality is a sequencing depth.

29. The method of claim 28, wherein the sequencing depth is between 1× and 500×.

30. The method of claim 28, wherein the sequencing depth is between 10× and 100×.

31. The method of claim 28, wherein the sequencing depth is greater than 1×.

32. The method of claim 28, wherein the sequencing depth is 10×, 20×, 30×, 40×, 50×, 60×, 70×, 80×, 90×, 100×, 110×, 120×, 130×, 140×, 150×, 160×, 170×, 180×, 190× or 200×.

33. A system for genetic sequencing, the system comprising:
a device for receiving a genetic sample;
a device for sequencing one or more portions of the genetic sample;
a device for capturing sequencing data;
at least one processor configured to:
generate signals for commencing sequencing the one or more portions of the genetic sample;
during or after sequencing of the one or more portions, receiving sequencing data for the one or more portions;
generating from the sequencing data, at least one sequencing quality metric, said at least one sequencing quality metric belonging to at least one category of sequencing quality metrics; and
generating signals for continuing or aborting the sequencing of the same or additional portions of the genetic sample based on a determination of the amount of sequencing of the genetic sample required to achieve a target sequencing quality using said at least one sequencing quality metric and a model trained with reference sequencing quality metrics to predict sequencing quality.

34.-43. (canceled)

44. A system for genome sequencing, the system comprising:
a device for receiving a genome sample;
a device for sequencing one or more portions of the genome sample;
a device for capturing sequencing data;
at least one processor configured to perform the method of claim 1.
